(19) United States
(12) Patent Application Publication
(10) Pub. No.: US 2005/0025808 A1
(43) Pub. Date: Feb. 3, 2005

(54) Medical devices and methods for inhibiting smooth muscle cell proliferation

(76) Inventors: Robert A. Herrmann, Boston, MA (US); James J. Barry, Marlborough, MA (US); Kalpana R. Kamath, Natick, MA (US)

Correspondence Address: JONES DAY, 222 EAST 41ST ST, NEW YORK, NY, 10017, US

(21) Appl. No.: 10/882,070

(22) Filed: Jun. 29, 2004

Related U.S. Application Data
(63) Continuation-in-part of application No. 10/252,848, filed on Sep. 24, 2002.
(60) Provisional application No. 60/324,095, filed on Sep. 24, 2001.
(60) Provisional application No. 60/483,820, filed on Jun. 30, 2003.

(51) Int. Cl. A61F 2/06

(52) U.S. Cl. 424/426

(57) ABSTRACT

The present invention relates generally to medical devices, preferably a stent, comprising an amount of one or more therapeutic agents, preferably paclitaxel, useful for preventing or treating a disease or condition associated with cell proliferation and/or migration. In particular, the invention relates to medical devices that is capable of releasing a cytostatic amount of paclitaxel that is effective to arrest smooth muscle cells in their G1/S phase without killing the cells. The medical devices can also release paclitaxel at a predetermined rate. Methods of making and using the medical devices are also provided.

This application claims priority benefit to U.S. Provisional Application No. 60/483,820, filed Jun. 30, 2003, and is a continuation-in-part of U.S. application Ser. No. 10/252,848, filed Sep. 24, 2002, which claims priority benefit to U.S. Provisional Application No. 60/324,095, filed Sep. 24, 2001, all of which are incorporated herein by reference in their entireties.

### 1. FIELD OF THE INVENTION

The present invention relates generally to methods of arresting smooth muscle cells in the G1/S phase of cell cycle by exposing the cells to low concentrations of an anti-proliferative agent such as paclitaxel. The present invention further relates to drug-eluting medical devices that are capable of providing sustained release of one or more therapeutic agents, preferably paclitaxel, over a time period and in an amount effective to inhibit smooth muscle cell proliferation and/or migration by arresting the cells in their G1/S phase. In one embodiment, the medical device is coated or covered with a surface coating comprising a polymeric material incorporating paclitaxel. Preferably, the polymeric material is biostable and may optionally comprise one or more biologically active materials, preferably a smooth muscle cell inhibitor and/or antibiotic. The invention also relates to methods of making and methods of using the drug-eluting medical device. The invention further relates to methods of preventing or treating a proliferative disease such as restenosis, stenosis, psoriasis, dermatitis, liver sclerosis, or benign prostate hyperplasia, by administering to a subject in need thereof a cytostatic amount of paclitaxel.

### 2. BACKGROUND OF THE INVENTION

Cardiovascular disease is a leading cause of death in the developed world. Patients having such disease usually have narrowing or closing (stenosis) in one or more arteries. The use of stents in the treatment of cardiovascular disease is well known. Stents are typically delivered in a contracted state to the treatment area within a lumen, where they are then expanded. Balloon-expandable stents expand from a contracted state by deforming in response to a force exerted upon the stent body by a balloon that is inflated within the stent's lumen. Once expanded within a body lumen, the stent body is strong enough to resist any contracting force exerted by the body lumen wall so that the stent maintains its expanded diameter. In contrast, self-expanding stents have resilient bodies that exert a radial expansion force when the stent is compressed. A self-expanding stent that is deployed within a body lumen will expand until the body lumen wall exerts a compressive force against the stent that is equal to the radial expansion force.

The use of balloon-expandable and self-expanding stents, however, may have the disadvantage of causing additional trauma to a body lumen upon deployment of the stent. Typically, a stent is expanded within a body lumen so that the diameter of the stent is greater than that of the body lumen. As a result, the edges of the ends of stent may be pressed into the wall of body lumen, stressing the wall to the point of creating additional trauma, i.e., cutting or tearing of the body lumen wall. This trauma may ultimately lead to restenosis, which is a re-narrowing of the coronary artery in the stented segment primarily due to vascular smooth muscle cell (SMC) proliferation. Restenosis occurs on average in 25% of patients receiving a stent (Kuntz R. E. et al. Prevention of coronary restenosis: the evolving evidence base for radiation therapy. Circulation. 2000 May 9;101(18):2130-3).

Recently, various types of drug-coated stents have been used for the localized delivery of drugs to the wall of a body lumen to further prevent restenosis. One strategy recently employed to prevent SMC proliferation is the use of stents that have been designed to release anti-proliferative drugs such as sirolimus (Sousa J. E. et al. Two-year angiographic and intravascular ultrasound follow-up after implantation of sirolimus-eluting stents in human coronary arteries. Circulation. 2003 Jan. 28;107(3):381-3) and paclitaxel (Axel D. I. et al. Paclitaxel inhibits arterial smooth muscle cell proliferation and migration in vitro and in vivo using local drug delivery. Circulation. 1997 Jul. 15;96(2):636-45; Garza L. et al. Can we prevent in-stent restenosis? Curr Opin Cardiol. 2002 Sep.;17(5):518-25; Grube E. et al. TAXUS I: six- and twelve-month results from a randomized, double-blind trial on a slow-release paclitaxel-eluting stent for de novo coronary lesions. Circulation. 2003 Jan. 7;107(1):38-42; Ferguson J. E. et al. Break the cycle: the role of cell-cycle modulation in the prevention of vasculoproliferative diseases. Cell Cycle. 2003 May-Jun.;2(3):211-9; Park S. J. et al. A paclitaxel-eluting stent for the prevention of coronary restenosis. N Engl J Med. 2003 Apr. 17;348(16):1537-45).

Paclitaxel is the active component of the anti-neoplastic drug Taxol®, which has been shown to be highly effective in a wide range of malignancies (Rowinsky E. K. et al. Paclitaxel (taxol). N Engl J Med. 1995 Apr. 13;332(15):1004-14; Rowinsky E. K. Update on the antitumor activity of paclitaxel in clinical trials. Ann Pharmacother. 1994 May;28(5 Suppl):S18-22; Blagosklonny M. V. Antimicrotuble agents. Cancer Handbook. Nature Publishing Group, 2002; (Editor: Alison M. R.). pp.1323-32). Taxol® is currently FDA-approved for various indications, including advanced carcinoma of the ovary, adjuvant treatment of node-positive breast cancer, breast cancer after failure of combination chemotherapy for metastatic disease or relapse within 6 months of adjuvant chemotherapy, non-small cell lung cancer (in combination with cisplatin), and AIDS-related Kaposi's sarcoma.

At concentration levels applicable to anti-neoplastic therapy, paclitaxel's mode of action has been attributed to its interference with the assembly of mitotic spindle resulting in prolonged mitotic arrest, subsequently leading to the induction of apoptosis, a rapid programmed cell death associated with the activation of caspases (Bhalla K. et al. Taxol induces intemucleosomal DNA fragmentation associated with programmed cell death in human myeloid leukemia cells. Leukemia. 1993 Apr.;7(4):563-8; Li X. et al. Apoptotic cell death during treatment of leukemias. Leuk Lymphoma. 1994; 13 Suppl 1:65-70; Perkins C.L. et al. The role of Apaf-1, caspase-9, and bid proteins in etoposide- or paclitaxel-induced mitochondrial events during apoptosis. Cancer Res. 2000 Mar. 15;60(6):1645-53; Blagosklonny M. V. et al. Mitogen-activated protein kinase pathway is dispensable for microtubule-active drug-induced Raf-1/Bcl-2 phosphorylation and apoptosis in leukemia cells. Leukemia. 1999 Jul.; 13(7):1028-36). Due to the cell death associated with paclitaxel at these high concentrations, it is considered to be a cytotoxic drug in the field of oncology.

Given that the effects of therapeutic agents may vary widely depending on the cell types, drug concentration, duration of drug exposure, and the microenvironment of the target cell population, it becomes imperative that effects of anti-stenotic/restenotic agents (e.g., paclitaxel) be investigated in cell populations relevant to the pathology under consideration rather than extrapolating results from its use in other disease conditions. The rapid adoption of drug-eluting stent technologies makes it all the more crucial to develop this understanding, so that clinical end-points can be effectively coupled to the underlying impact of drugs at the cellular level.

### 3. SUMMARY OF THE INVENTION

The present inventors discovered a novel mechanism of paclitaxel action for the treatment of restenosis following coronary stent implantation. Specifically, the inventors have made the discovery that when exposed to relatively low concentrations of paclitaxel, smooth muscle cells are arrested in the G1/S phase of the cell cycle, without inducing the apoptotic pathway. This provides a novel cytostatic mechanism for inhibiting proliferation of smooth muscle cells following placement of paclitaxel-eluting stent in the coronary artery.

In certain embodiments, the present invention relates to a method of arresting smooch muscle cells in their G1/S phase by exposing the cells to a concentration of paclitaxel that is about 0.001 ng/ml to about 10,000 ng/ml, preferably about 0.01 ng/ml to about 1,000 ng/ml, or about 60 ng/ml to about 6,000 ng/ml. In a specific embodiment, most (i.e., greater than about 80%, preferably about 90% to about 100%) of the smooth muscle cells are arrested in the G1/S phase of the cell cycle when exposed to low concentrations of paclitaxel.

In certain embodiments, the present invention relates to paclitaxel-eluting medical devices that can be inserted or implanted into a body lumen comprising smooth muscle cells. The medical device comprises a surface and a surface coating that comprises a polymeric material incorporating paclitaxel. In a preferred embodiment, the surface of the medical device is coated with a polymeric material that comprises about 0.5 μg to about 5 μg paclitaxel per mm² of the surface area of the surface. In a more preferred embodiment, the surface of the medical device is coated with a polymeric material that comprises about 1 μg paclitaxel per mm² of the surface area of the surface.

In another preferred embodiment, the polymeric material that is used to coat the surface of the medical device is biostable. In another preferred embodiment, the polymeric material that is used to coat the surface of the medical device comprises a styrene-isobutylene copolymer. In another preferred embodiment, the polymeric material that is used to coat the surface of the medical device comprises a biologically active material in addition to paclitaxel.

In a specific embodiment, the medical device is capable of releasing an amount of the paclitaxel incorporated in the polymeric material of the surface coating that is effective to arrest most smooth muscle cells that are exposed to the released paclitaxel in the G1/S phase of the cell cycle. Preferably, about 90% to about 100% of the smooth muscle cells that are exposed to paclitaxel are arrested.

In yet another embodiment, the medical device is capable of releasing about 0.001 μg to about 20 μg of paclitaxel per mm² of the surface area of the surface over about 1 week to about 8 weeks. In a preferred embodiment, the medical device is capable of releasing about 0.01 μg to about 0.1 μg of paclitaxel per mm² of the surface area of the surface over about 4 weeks. In another preferred embodiment, the medical device is capable of releasing 0.1% to about 35%, more preferably 1% to about 15%, of the paclitaxel incorporated in the polymeric material over about 1 week to about 8 weeks. Preferably, the medical device is capable of continuously releasing paclitaxel for over 4 weeks.

In one embodiment, the amount of paclitaxel released by the medical device exposes the smooth muscle cells to a concentration of paclitaxel that is about 0.001 ng/ml to about 10,000 ng/ml, preferably about 0.01 ng/ml to about 1,000 ng/ml, or about 60 ng/ml to about 6,000 ng/ml.

The medical device described herein can be used to prevent or treat stenosis or restenosis in a subject. Preferably, the subject is a human.

In certain other embodiments, the present invention relates to a method for treating a proliferative disease by administering to a subject in need thereof a therapeutically effective amount of paclitaxel. In a preferred embodiment, the therapeutically effect amount is about 0.001 ng/ml to about 10,000 ng/ml. In a more preferred embodiment, the therapeutically effect amount is about 0.01 ng/ml to about 1,000 ng/ml. In a preferred embodiment, the therapeutically effect amount is about 60 ng/ml to about 6,000 ng/ml.

The paclitaxel can be administered to the subject by parenteral, subcutaneous, intramuscular, intraorbital, intracapsular, intraspinal, intrastemal, intravenous, intradermal, intraperitoneal, intraportal, intra-arterial, intrathecal, transmucosal, intra-articular, intrapleural, transdermal, topical, epidural, mucosal, intranasal injection or infusion, or oral, inhalation, pulmonary or rectal administration. In a specific embodiment, the paclitaxel is directly administered into a body lumen of the subject that comprises smooth muscle cells. Preferably, the therapeutically effective amount of paclitaxel is effective to arrest the smooth muscle cells of the body lumen in the G1/S phase of the cell cycle.

The method is useful for treating without limitation restenosis, stenosis, psoriasis, dermatitis, liver sclerosis, and benign prostate hyperplasia.

### 4. FIGURES

FIG. 1. Effects of PTX on Human Arterial SMC Proliferation.

FIGS. 1A. Human arterial smooth muscle cells (hSMC) were treated with indicated concentrations (0.001-10,000 ng/ml) of paclitaxel (PTX) or Taxol® for 5 days and then counted. Results were calculated as the percent of values obtained with control untreated cells. FIG. 1B. Human arterial smooth muscle cells were treated with the indicated concentrations of PTX for 3 and 4 days. The MTT (3-(4,5-dimethylthiazol-2-yl)-2,5-diphenyltetrazolium bromide) cell proliferation assay was performed as described by the methods of Section 6 infra. Results were calculated as the percent of values obtained with untreated cells and represent mean ±standard deviation (SD). FIG. 1C. Human smooth muscle cells were incubated with 2 or 12 ng/ml PTX or left untreated (control). At days indicated, the percentage of live cells with respect to control was calculated using trypan blue exclusion, as described by the methods of Section 6 infra.

FIG. 2. Interphase Cells, Mitotic cells (M), Multinucleated (MN) Cells and DNA Distribution

Human smooth muscle cells were treated with 0, 2, 12, 60 ng/ml PTX, as indicated, for two days, DAPI (4′-6-diamidino-2-phenylindole) staining (FIG. 2A) and flow cytometry (FIG. 2B) were performed as described by the methods of Section 6 infra.

FIG. 3. Cellular and Nuclear Morphology of hSMC Following PTX Treatment.

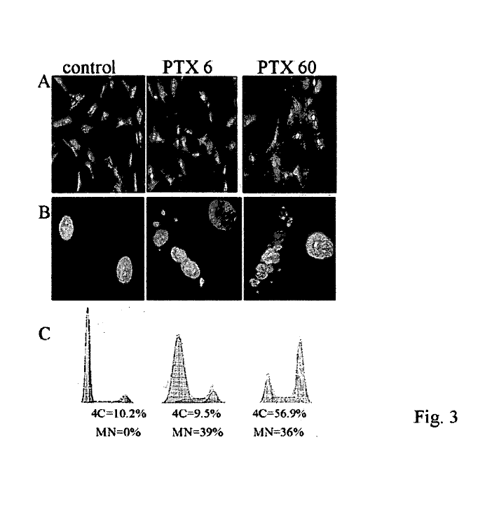

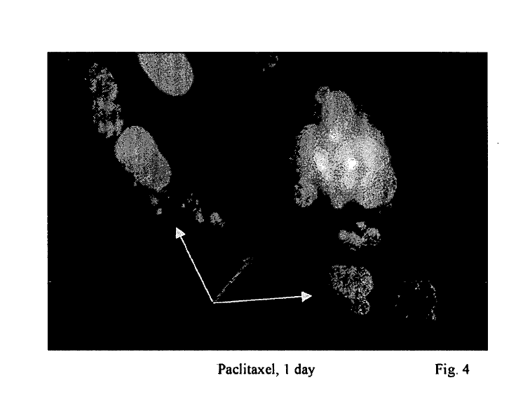

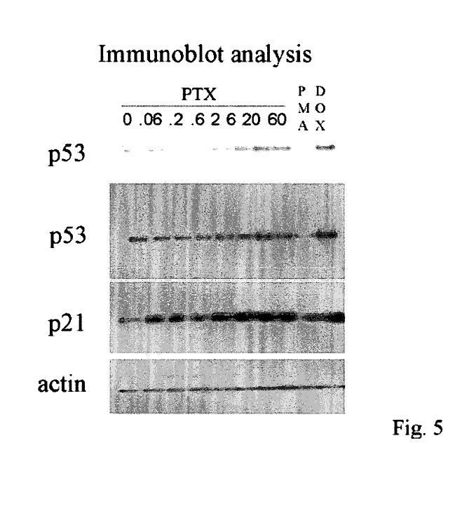

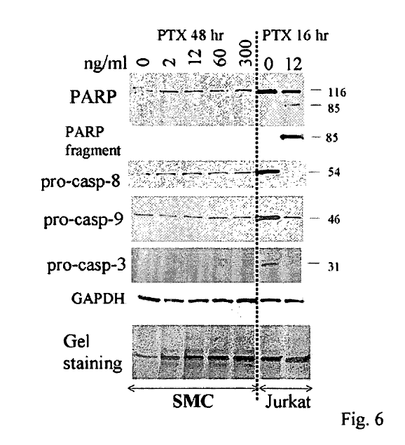

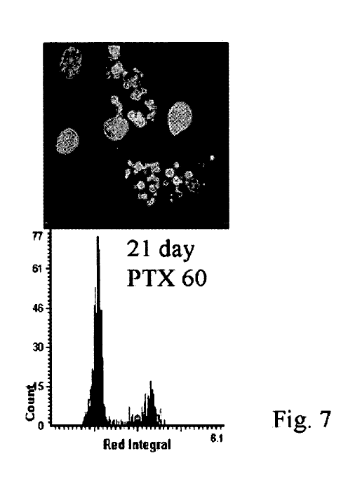

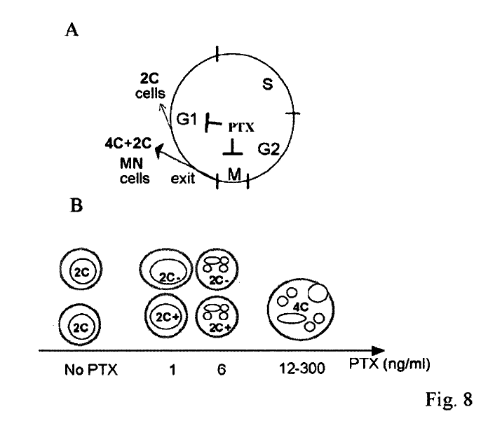

Human smooth muscle cells were cultured on slides and treated with indicated concentrations of PTX (6 ng/ml, 60 ng/ml, or untreated). Following 24 hrs, cells were fixed and nucleus (red/yellow) and cytoplasm (green) were stained (Rhodamine/FITC) as described by the methods of Section 6 infra. FIG. 3A. Photomicrographs indicate normal smooth muscle cell morphology in the non-treated cells and cells treated with 6 and 60 ng/ml of paclitaxel. FIG. 3B. Images of the nuclei demonstrate normal nuclei with no paclitaxel treatment and multi-nuclei with paclitaxel treatment at 6 and 60 ng/ml. FIG. 3C. After 2 days, cells were analyzed by flow cytometry (DNA content) and by DAPI staining (numbers of MN cells).

FIG. 4. DNA Distribution between Dividing MN Cells

Human smooth muscle cells were grown on slides and treated with 6 ng/ml PTX. After 1 day, cells were fixed and DNA was stained (red) as described in Methods. Photo of the nuclei were taken at 200×.

FIG. 5. Effects of PTX on p53 and p21 Levels

Human smooth muscle cells were treated either with indicated concentrations of PTX, with 400 ng/ml Doxorubicin (DOX) (a positive control), or with 100 ng/ml phorbol 12-myristate 13-acetate (PMA) (negative control for p53). After 24 hr, cells were lysed and p21 and p53 were determined by immunoblot as described in Section 6 infra. (Note: For p53, upper and second panels are the same blot with different film exposure time).

FIG. 6. Effects of PTX on Caspase Cleavage (Apoptosis)

Human smooth muscle cells and Jurkat cells were incubated with indicated concentrations of PTX. After 16 and 48 hours, Jurkat and human smooth muscle cells (respectively) were lysed and poly(ADP-ribose) polymerase (PARP), PARP 89 kDa fragment, procaspase-8, -9 and -3 were identified via immunoblot described by the methods of Section 6 infra. Glyceraldehyde phosphate dehydrogenase (GAPDH) immunoblot analysis and gel staining were performed as a loading controls.

FIG. 7. Long Term Survival of SMC

Human smooth muscle cells were incubated with 60 ng/ml PTX for 21 days, and analyzed by nuclear staining (top panel) and flow cytometry (bottom panel).

FIG. 8. Fate of SMC following PTX Exposure

FIG. 8A. Points of cell cycle arrest. All human smooth muscle cells were eventually arrested in G1. Human smooth muscle cells that are in G1 at the time of PTX exposure may be arrested in G1 (primary G1 arrest). All other human smooth muscle cells continue the cell cycle and enter mitosis. Following abnormal mitotic exit, multinucleated human smooth muscle cells were arrested in G1. FIG. 8B. Following mitotic exit, human smooth muscle cells may form 2C (normal) cells, 2C multinucleated or single nucleus cells or multinucleated 4C cells, depending on PTX concentrations.

### 5. DETAILED DESCRIPTION OF THE INVENTION

The present invention relates generally to methods of arresting smooth muscle cells in the G1/S phase of the cell cycle. The present invention further relates to drug-eluting medical devices that are capable of releasing one or more therapeutic agents in an amount that is effective to inhibit smooth muscle cell proliferation and/or migration by arresting most of the cells exposed to the released therapeutic agent(s) in their G1/S phase. In certain embodiments, the medical device is coated or covered with one or more drug-eluting coatings comprising one or more polymeric materials incorporating one or more therapeutic agents. In certain other embodiments, the medical device is made of one or more drug-eluting polymer mixture comprising one or more polymeric materials incorporating one or more therapeutic agents.

In one embodiment, the medical device comprises a surface coating comprising a polymeric material incorporating paclitaxel. In a specific embodiment, the medical device is coated or covered with a surface coating comprising about 0.5 μg to about 5 μg of paclitaxel per cm² of the surface area of the surface of the medical device. In a preferred embodiment, the medical device is coated or covered with a surface coating comprising about 1 μg of paclitaxel per mm² of the surface area of the surface of the medical device.

In another embodiment, the medical device is capable of releasing a cytostatic amount or percentage of the paclitaxel incorporated in the polymeric material that makes up the surface coating over a period of time. As used herein, a “cytostatic” amount can mean, but is not limited to, an amount that does not kill the cells and/or inhibits DNA synthesis more than it inhibits protein synthesis.

In yet another embodiment, the medical device is capable of releasing about 0.001 μg to about 20 μg of paclitaxel per mm² surface area of the surface over about 1 week to about 8 weeks. In a preferred embodiment, the medical device is capable of releasing about 0.01 μg to about 0.1 μg of paclitaxel per mm² surface area of the surface over about 4 weeks. In another preferred embodiment, the medical device is capable of releasing about 0.1% to about 35% of the paclitaxel incorporated in the polymeric material that makes up the surface coating. Preferably, the medical device is capable of releasing about 0.1% to about 15% of the paclitaxel over 10 days in an in vitro environment.

Preferably, the medical device is capable of releasing an amount of paclitaxel such that the circulating levels of the released paclitaxel is maintained at least at about 0.001 ng/ml to about 10,000 ng/ml for about 1 week to about 8 weeks.

Preferably, the target cells are exposed to a concentration of paclitaxel that is capable of arresting most of the cells in the G1/S phase. Preferably, the concentration of paclitaxel that the cells are exposed to does not induce apoptotic cell death. Preferably, the concentration of paclitaxel that the cells are exposed to is about 0.001 ng/ml to about 10,000 ng/ml. More preferably, the concentration of paclitaxel that the cells are exposed to is about 0.01 ng/ml to about 1,000 ng/ml. Preferably, the concentration of paclitaxel that the cells are exposed to is about 60 ng/ml to about 6,000 ng/ml. As used herein, “paclitaxel” refers to paclitaxel, its analogs and its derivatives.

The invention also relates to methods of treating or preventing stenosis or restenosis by inserting or implanting the drug-eluting medical devices into a subject in need thereof. The invention further relates to methods of treating or preventing disease or condition associated with cell proliferation and/or migration (“a proliferative disease”), such as restenosis, stenosis, psoriasis, dermatitis, liver sclerosis, and benign prostate hyperplasia, by administering to a subject in need thereof a therapeutically effective amount of paclitaxel. Specifically, the therapeutically effective amount of paclitaxel is a cytostatic amount of paclitaxel that arrests most (ie., more than 80%), preferably about 90% to about 100%, of the smooth muscle cells in the G1/S phase of the cell cycle without inducing apoptotic cell death.

For clarity of disclosure, and not by way of limitation, the detailed description of the invention is divided into the subsections which follow.

5.1 Methods For Making The Medical Devices

5.1.1 Drug-Eluting Coatings

Coating compositions suitable for forming coatings for the devices of the present invention can include one or more therapeutic agents as described in Section 5.1.2 infra. and one or more polymeric materials as described in Section 5.1.3 infra. In certain embodiments, the coating compositions comprise one, two, three, four, five or more polymeric materials. In certain embodiments, the polymeric materials comprises one, two, three, four, five or more therapeutic agents.

In one embodiment, the coating composition comprises a polymeric material incorporating a therapeutic agent, preferably paclitaxel. The polymeric material incorporates the paclitaxel or other therapeutic agent by intermixing with the paclitaxel or therapeutic agent, e.g., the polymeric material surrounds at least some of the paclitaxel or therapeutic agent. Optionally, the coating can comprise one or more additional therapeutic agents. In one embodiment, the coating comprises a first polymeric material comprising a first therapeutic agent and a second polymeric material comprising a second therapeutic agent. In a specific embodiment, the first and second therapeutic agents are the same, e.g., paclitaxel. In another specific embodiment, the first and second therapeutic agents are different, e.g., paclitaxel and rapamycin.

To prepare the coating compositions, the constituents, e.g., polymer, paclitaxel, and optionally an additional therapeutic agent, are suspended and/or dissolved in a solvent. Preferably, the solvent does not alter or adversely impact the therapeutic properties of the therapeutic agent(s) employed. For example, useful solvents for paclitaxel include polyethoxylated castor oil such as Cremophor® EL solution. Inclusion of both the polymeric material and paclitaxel in the coating composition forms a coating wherein the polymeric material incorporates the paclitaxel.

In specific embodiments, the coating composition comprises at least about 5%, at least about 10%, at least about 20%, at least about 30%, at least about 40%, at least about 50%, at least about 60%, at least about 70%, at least about 80%, at least about 90%, at least about 95%, at least about 97%, at least about 99% or more by weight of the polymeric material. In specific embodiments, the coating composition comprises at least about 5%, at least about 10%, at least about 20%, at least about 30%, at least about 40%, at least about 50%, at least about 60%, at least about 70%, at least about 80%, at least about 90%, at least about 95%, at least about 97%, at least about 99% or more by weight of a (first) therapeutic agent, preferably paclitaxel. In specific embodiments, the coating composition comprises at least about 5%, at least about 10%, at least about 20%, at least about 30%, at least about 40%, at least about 50%, at least about 60%, at least about 70%, at least about 80%, at least about 90%, at least about 95%, at least about 97%, at least about 99% or more by weight of the additional (second, third, fourth, or fifth) therapeutic agent(s).

In a specific embodiment, the coating composition comprises about 0.001 μg, about 0.01 μg, about 0.1 μg, about 1 μg, 5 μg, about 10 μg, about 15 μg, about 20 μg, about 25 about 30 μg, about 35 μg, about 40 μg, about 45 μg, about 50 μg, about 60 μg, about 70 μg, about 80 μg, about 90 μg, about 100 μg, about 110 μg, about 120 μg, about 130 μg, about 140 μg, about 150 μg, about 200 μg, about 250 μg, about 300 μg, about 350 μg, about 400 μg, about 500 μg, about 600 μg, about 700 μg, about 800 μg, about 900 μg, about 1,000 μg, about 2,000 μg or more of the therapeutic agent. Preferably, the coating composition comprises about 50 μg to about 200 μg paclitaxel.

In another specific embodiment, the coating composition comprises about 0.001 μg, about 0.01 μg, about 0.1 μg, about 0.5 μg, about 1.0 μg, about 2.0 μg, about 3.0 μg, about 4.0 μg, about 5.0 μg, about 6.0 μg, about 7.0 μg, about 8.0 μg, about 9.0 μg, about 10.0 μg, about 15.0 μg, about 20.0 μg or more of the therapeutic agent per mm² of the surface area of the surface of the medical device. Preferably, the coating composition comprises about 0.5 μg to about 5 μg paclitaxel per mm² of the surface area of the surface of the medical device.

In certain embodiments, the coating composition is capable of releasing a cytostatic amount of a therapeutic agent that is effective of freezing the cell in the G1/S phase.

In one embodiment, the coating composition releases about 0.1%, about 1%, about 5%, about 10%, about 15%, about 20%, about 25%, about 30%, about 35%, about 40%, about 45%, about 50%, about 55%, about 60%, about 65%, about 70%, about 75%, about 80%, about 90%, about 95% or more of the paclitaxel incorporated in the polymeric material over about 30 minutes, 1 hour, 2 hours, 6 hours, about 12 hours, about 24 hours, about 2 days, about 3 days, about 4 days, about 5 days, about 6 days, about 1 week, about 2 weeks, about 3 weeks, about 1 month, about 2 months, about 3 months, about 4 months, about 5 months, about 6 months, about 1 year, about 2 years, about 5 years, etc. Preferably, the coating composition is capable of releasing about 0.1% to about 35% of the amount of the paclitaxel incorporated in the polymeric material over about 1 week to about 8 weeks. More preferably, the coating composition is capable of releasing about 1% to about 15% of the amount of the paclitaxel incorporated in the polymeric material over about 4 weeks.

In another embodiment, the coating composition releases about 0.001 μg, about 0.01 μg, about 0.1 μg, about 1 μg, 5 μg, about 10 μg, about 15 μg, about 20 μg, about 25 μg, about 30 μg, about 35 μg, about 40 μg, about 45 μg, about 50 μg, about 60 μg, about 70 μg, about 80 μg, about 90 μg, about 100 μg, about 110 μg, about 120 μg, about 130 μg, about 140 μg, about 150 μg, about 200 μg, about 250 μg, about 300 μg, about 350 μg, about 400 μg, about 500 μg, about 600 μg, about 700 μg, about 800 μg, about 900 μg, about 1,000 μg, about 2,000 μg or more of the therapeutic agent over about 30 minutes, 1 hour, 2 hours, 6 hours, about 12 hours, about 24 hours, about 2 days, about 3 days, about 4 days, about 5 days, about 6 days, about 1 week, about 2 weeks, about 3 weeks, about 1 month, about 2 months, about 3 months, about 4 months, about 5 months, about 6 months, about 1 year, about 2 years, about 5 years, etc. Preferably, the coating composition is capable of releasing about 50 μg to about 200 μg of paclitaxel incorporated in the polymeric material over about 1 week to about 8 weeks.

In yet another embodiment, the coating composition releases about 0.001 μg, about 0.01 μg, about 0.1 μg, about 0.5 μg, about 1.0 μg, about 2.0 μg, about 3.0 μg, about 4.0 μg, about 5.0 μg, about 6.0 μg, about 7.0 μg, about 8.0 μg, about 9.0 μg, about 10.0 μg, about 15.0 μg, about 20.0 μg or more of the therapeutic agent per mm² of the surface area of the surface of the medical device over about 30 minutes, 1 hour, 2 hours, 6 hours, about 12 hours, about 24 hours, about 2 days, about 3 days, about 4 days, about 5 days, about 6 days, about 1 week, about 2 weeks, about 3 weeks, about 1 month, about 2 months, about 3 months, about 4 months, about 5 months, about 6 months, about 1 year, about 2 years, about 5 years, etc. Preferably, the coating composition is capable of releasing about 0.01 μg to about 0.1 μg of paclitaxel incorporated in the polymeric material over about 1 week to about 8 weeks.

In certain other embodiments, the coating composition is capable of continuously releasing therapeutic agent over a period of time and thereby exposing the cells to a concentration of therapeutic agent that is effective of freezing the cell in the G1/S phase. Preferably, the concentration of therapeutic agent that the cells is exposed to is about 0.0001 ng/ml, about 0.001 ng/ml, about 0.01 ng/ml, about 0.1 ng/ml, about 1.0 ng/ml, about 10 ng/ml, about 20 ng/ml, about 30 ng/ml, about 40 ng/ml, about 50 ng/ml, about 100 ng/ml, about 200 ng/ml, about 300 ng/ml, about 400 ng/ml, about 500 ng/ml, about 600 ng/ml, about 700 ng/ml, about 800 ng/ml, about 900 ng/ml, about 1,000 ng/ml, about 2,000 ng/ml, about 3,000 ng/ml, about 4,000 ng/ml, about 5,000 ng/ml, about 10,000 ng/ml or more of the one or more therapeutic agents. Preferably, the concentration of therapeutic agent that the cells is exposed to is about 0.001 ng/ml to 10,000 ng/ml of paclitaxel. More preferably, the concentration of therapeutic agent that the cells is exposed to is about 0.01 ng/ml to 1,000 ng/ml of paclitaxel. Preferably, the concentration of therapeutic agent that the cells is exposed to is about 60 ng/ml to about 6,000 ng/ml.

5.1.2 Therapeutic Agents/Biologically Active Materials

The term “therapeutic agent” as used in the present invention encompasses drugs, genetic materials, and biological materials and can be used interchangeably with “biologically active material”. Non-limiting examples of suitable therapeutic agent include heparin, heparin derivatives, urokinase, dextrophenylalanine proline arginine chloromethylketone (PPack), enoxaprin, angiopeptin, hirudin, acetylsalicylic acid, tacrolimus, everolimus, rapamycin (sirolimus), amlodipine, doxazosin, glucocorticoids, betamethasone, dexamethasone, prednisolone, corticosterone, budesonide, sulfasalazine, rosiglitazone, mycophenolic acid, mesalamine, paclitaxel, 5-fluorouracil, cisplatin, vinblastine, vincristine, epothilones, methotrexate, azathioprine, adriamycin, mutamycin, endostatin, angiostatin, thymidine kinase inhibitors, cladribine, lidocaine, bupivacaine, ropivacaine, D-Phe-Pro-Arg chloromethyl ketone, platelet receptor antagonists, anti-thrombin antibodies, anti-platelet receptor antibodies, aspirin, dipyridamole, protamine, hirudin, prostaglandin inhibitors, platelet inhibitors, trapidil, liprostin, tick antiplatelet peptides, 5-azacytidine, vascular endothelial growth factors, growth factor receptors, transcriptional activators, translational promoters, antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin, cholesterol lowering agents, vasodilating agents, agents which interfere with endogenous vasoactive mechanisms, antioxidants, probucol, antibiotic agents, penicillin, cefoxitin, oxacillin, tobranycin, angiogenic substances, fibroblast growth factors, estrogen, estradiol (E2), estriol (E3), 17-beta estradiol, digoxin, beta blockers, captopril, enalopril, statins, steroids, vitamins, taxol, paclitaxel, 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl) glutamine, 2′-O-ester with N-(dimethylaminoethyl) glutamide hydrochloride salt, nitroglycerin, nitrous oxides, nitric oxides, antibiotics, aspirins, digitalis, estrogen, estradiol and glycosides. In one embodiment, the therapeutic agent is a smooth muscle cell inhibitor or antibiotic. In a preferred embodiment, the therapeutic agent is taxol (e.g., Taxol®), or its analogs or derivatives. In another preferred embodiment, the therapeutic agent is paclitaxel, or its analogs or derivatives. In yet another preferred embodiment, the therapeutic agent is an antibiotic such as erythromycin, amphotericin, rapamycin, adriamycin, etc.

The term “genetic materials” means DNA or RNA, including, without limitation, of DNA/RNA encoding a useful protein stated below, intended to be inserted into a human body including viral vectors and non-viral vectors.

The term “biological materials” include cells, yeasts, bacteria, proteins, peptides, cytokines and hormones. Examples for peptides and proteins include vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factors (CGF), platelet-derived growth factor (PDGF), hypoxia inducible factor-1 (HIF- 1), stem cell derived factor (SDF), stem cell factor (SCF), endothelial cell growth supplement (ECGS), granulocyte macrophage colony stimulating factor (GM-CSF), growth differentiation factor (GDF), integrin modulating factor (IMF), calmodulin (CaM), thymidine kinase (TK), tumor necrosis factor (TNF), growth hormone (GH), bone morphogenic protein (BMP) (e.g., BMP-2, BMP-3, BMP-4, BMP-5, BMP-6 (Vgr-1), BMP-7 (PO-1), BMP-8, BMP-9, BMP-10, BMP-11, BMP-12, BMP-14, BMP-15, BMP-16, etc.), matrix metalloproteinase (MMP), tissue inhibitor of matrix metalloproteinase (TIMP), cytokines, interleukin (e.g., IL-1, IL-2, IL-3, IL-4, IL-5, IL-6, IL-7, IL-8, IL-9, IL-10, IL-11, IL-12, IL-15, etc.), lymphokines, interferon, integrin, collagen (all types), elastin, fibrillins, fibronectin, vitronectin, laminin, glycosaminoglycans, proteoglycans, transferrin, cytotactin, cell binding domains (e.g., RGD), and tenascin. Currently preferred BMP's are BMP-2, BMP-3, BMP-4, BMP-5, BMP-6, BMP-7. These dimeric proteins can be provided as homodimers, heterodimers, or combinations thereof, alone or together with other molecules. Cells can be of human origin (autologous or allogeneic) or from an animal source (xenogeneic), genetically engineered, if desired, to deliver proteins of interest at the transplant site. The delivery media can be formulated as needed to maintain cell function and viability. Cells include progenitor cells (e.g., endothelial progenitor cells), stem cells (e.g., mesenchymal, hematopoietic, neuronal), stromal cells, parenchymal cells, undifferentiated cells, fibroblasts, macrophage, and satellite cells.

Other non-genetic therapeutic agents include:
anti-thrombogenic agents such as heparin, heparin derivatives, urokinase, and PPack (dextrophenylalanine proline arginine chloromethylketone);
anti-proliferative agents such as enoxaprin, angiopeptin, or monoclonal antibodies capable of blocking smooth muscle cell proliferation, hirudin, acetylsalicylic acid, tacrolimus, everolimus, amlodipine and doxazosin;
anti-inflammatory agents such as glucocorticoids, betamethasone, dexamethasone, prednisolone, corticosterone, budesonide, estrogen, sulfasalazine, rosiglitazone, mycophenolic acid and mesalamine;
anti-neoplastic/anti-proliferative/anti-miotic agents such as paclitaxel, 5-fluorouracil, cisplatin, vinblastine, vincristine, epothilones, methotrexate, azathioprine, adriamycin and mutamycin; endostatin, angiostatin and thymidine kinase inhibitors, cladribine, taxol and its analogs or derivatives;
anesthetic agents such as lidocaine, bupivacaine, and ropivacaine;
anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors, antiplatelet agents such as trapidil or liprostin and tick antiplatelet peptides;
DNA demethylating drugs such as 5-azacytidine, which is also categorized as a RNA or DNA metabolite that inhibit cell growth and induce apoptosis in certain cancer cells;
vascular cell growth promoters such as growth factors, vascular endothelial growth factors (VEGF, all types including VEGF-2), growth factor receptors, transcriptional activators, and translational promoters;
vascular cell growth inhibitors such as anti-proliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin;
cholesterol-lowering agents, vasodilating agents, and agents which interfere with endogenous vasoactive mechanisms;
anti-oxidants, such as probucol;
antibiotic agents, such as penicillin, cefoxitin, oxacillin, tobranycin, rapamycin (sirolimus);
angiogenic substances, such as acidic and basic fibroblast growth factors, estrogen including estradiol (E2), estriol (E3) and 17-beta estradiol;
drugs for heart failure, such as digoxin, beta-blockers, angiotensin-converting enzyme (ACE) inhibitors including captopril and enalopril, statins and related compounds; and
macrolides such as sirolimus or everolimus.

Preferred biological materials include anti-proliferative drugs such as steroids, vitamins, and restenosis-inhibiting agents. Preferred restenosis-inhibiting agents include microtubule stabilizing agents such as Taxol®, paclitaxel (i.e., paclitaxel, paclitaxel analogs, or paclitaxel derivatives, and mixtures thereof). For example, derivatives suitable for use in the present invention include 2′-succinyl-taxol, 2′-succinyl-taxol triethanolamine, 2′-glutaryl-taxol, 2′-glutaryl-taxol triethanolamine salt, 2′-O-ester with N- (dimethylaminoethyl) glutamine, and 2′-O-ester with N-(dimethylaminoethyl) glutamide hydrochloride salt.

Other suitable therapeutic agents include tacrolimus, halofuginone, inhibitors of HSP90 heat shock proteins such as geldanamycin, microtubule stabilizing agents such as epothilone D, phosphodiesterase inhibitors such as cliostazole.

Other preferred therapeutic agents include nitroglycerin, nitrous oxides, nitric oxides, aspirins, digitalis, estrogen derivatives such as estradiol and glycosides.

In one embodiment, the therapeutic agent is capable of altering the cellular metabolism or inhibiting a cell activity, such as protein synthesis, DNA synthesis, spindle fiber formation, cellular proliferation, cell migration, microtubule formation, microfilament formation, extracellular matrix synthesis, extracellular matrix secretion, or increase in cell volume. In another embodiment, the therapeutic agent is capable of inhibiting cell proliferation and/or migration.

In certain embodiments, the therapeutic agents for use in the medical devices of the present invention can be synthesized by methods well known to one skilled in the art. Alternatively, the therapeutic agents can be purchased from chemical and pharmaceutical companies.

5.1.3 Polymeric Material

The polymeric material suitable for use in the preparation of the drug-eluting coatings of the present invention should be a material that is biocompatible and avoids irritation to body tissue. The polymeric materials can be biostable or bioabsorbable. Preferably, the polymeric material is biostable. Preferably, the polymeric materials used in the coating compositions of the present invention are selected from the following: polyurethanes, silicones (e.g., polysiloxanes and substituted polysiloxanes), and polyesters. Also preferable as a polymeric material are styrene-isobutylene copolymers. Other polymers which can be used include ones that can be dissolved and cured or polymerized on the medical device or polymers having relatively low melting points that can be blended with biologically active materials. Additional suitable polymers include, thermoplastic elastomers in general, polyolefins, polyisobutylene, ethylene-alphaolefin copolymers, acrylic polymers and copolymers, vinyl halide polymers and copolymers such as poly(lactide-co-glycolide) (PLGA), polyvinyl alcohol (PVA), poly(L-lactide) (PLLA), polyanhydrides, polyphosphazenes, polycaprolactone (PCL), polyvinyl chloride, polyvinyl ethers such as polyvinyl methyl ether, polyvinylidene halides such as polyvinylidene fluoride and polyvinylidene chloride, polyacrylonitrile, polyvinyl ketones, polyvinyl aromatics such as polystyrene, polyvinyl esters such as polyvinyl acetate, copolymers of vinyl monomers, copolymers of vinyl monomers and olefins such as ethylene-methyl methacrylate copolymers, acrylonitrile-styrene copolymers, ABS (acrylonitrile-butadiene-styrene) resins, ethylene-vinyl acetate copolymers, polyamides such as Nylon 66 and polycaprolactone, alkyd resins, polycarbonates, polyoxymethylenes, polyimides, polyethers, epoxy resins, rayon-triacetate, cellulose, cellulose acetate, cellulose butyrate, cellulose acetate butyrate, cellophane, cellulose nitrate, cellulose propionate, cellulose ethers, carboxymethyl cellulose, collagens, chitins, polylactic acid (PLA), polyglycolic acid (PGA), polyethylene oxide (PEO), polylactic acid-polyethylene oxide copolymers, EPDM (etylene-propylene-diene) rubbers, fluorosilicones, polyethylene glycol (PEG), polyalkylene glycol (PAG), polysaccharides, phospholipids, and combinations of the foregoing.

In certain embodiments, the polymeric material is hydrophilic (e.g., PVA, PLLA, PLGA, PEG, and PAG). In certain other embodiments, the polymeric material is hydrophobic (e.g., PLA, PGA, polyanhydrides, polyphosphazenes and PCL).

More preferably for medical devices which undergo mechanical challenges, e.g. expansion and contraction, the polymeric materials should be selected from elastomeric polymers such as silicones (e.g. polysiloxanes and substituted polysiloxanes), polyurethanes, thermoplastic elastomers, ethylene vinyl acetate copolymers, polyolefin elastomers, and EPDM rubbers. Because of the elastic nature of these polymers, the coating composition is capable of undergoing deformation under the yield point when the device is subjected to forces, stress or mechanical challenge.

In some embodiments, the polymeric materials are biodegradable. Biodegradable polymeric materials can degrade as a result of hydrolysis of the polymer chains into biologically acceptable, and progressively smaller compounds. In one embodiment, a polymeric material comprises polylactides, polyglycolides, or their co-polymers. Polylactides, polyglycolides, and their co-polymers break down to lactic acid and glycolic acid, which enters the Kreb's cycle and are further broken down into carbon dioxide and water.

The polymeric materials can also degrade through bulk hydrolysis, in which the polymer degrades in a fairly uniform manner throughout the matrix. For some novel degradable polymers, most notably the polyanhydrides and polyorthoesters, the degradation occurs only at the surface of the polymer, resulting in a release rate that is proportional to the surface area of the drug therapeutic agents and/or polymer/therapeutic agent mixtures. Hydrophilic polymeric materials such as PLGA will erode in a bulk fashion. Various commercially available PLGA may be used in the preparation of the coating compositions. For example, poly(d,1-lactic-co-glycolic acid) are commercially available. A preferred commercially available product is a 50:50 poly(d,1-lactic-co-glycolic acid) (d,1-PLA) having a mole percent composition of 50% lactide and 50% glycolide. Other suitable commercially available products are 65:35, 75:25, and 85:15 poly(d,1-lactic-co-glycolic acid). For example, poly(lactide-co-glycolides) are also commercially available from Boehringer Ingelheim (Germany) under the tradename Resomer®, e.g., PLGA 50:50 (Resomer RG 502), PLGA 75:25 (Resomer RG 752) and d,1-PLA (resomer RG 206), and from Birmingham Polymers (Birmingham, Alabama). These copolymers are available in a wide range of molecular weights and ratios of lactic to glycolic acid.

In one embodiment, the coating comprises copolymers with desirable hydrophilic/hydrophobic interactions (see, e.g., U.S. Pat. No. 6,007,845, which describes nanoparticles and microparticles of non-linear hydrophilic-hydrophobic multib lock copolymers, which is incorporated by reference herein in its entirety). In a specific embodiment, the coating comprises ABA triblock copolymers consisting of biodegradable A blocks from PLG and hydrophilic B blocks from PEO.

5.1.4 Types of Medical Device

Medical devices that are useful in the present invention can be made of any biocompatible material suitable for medical devices in general which include without limitation natural polymers, synthetic polymers, ceramics and metallics. Metallic material is more preferable. Suitable metallic materials include metals and alloys based on titanium (such as nitinol, nickel titanium alloys, thermo-memory alloy materials), stainless steel, tantalum, nickel-chrome, or certain cobalt alloys including cobalt-chromium-nickel alloys such as Elgiloy® and Phynox®. Metallic materials also include clad composite filaments, such as those disclosed in WO 94/16646.

Metallic materials may be made into elongated members or wire-like elements and then woven to form a network of metal mesh. Polymer filaments may also be used together with the metallic elongated members or wire-like elements to form a network mesh. If the network is made of metal, the intersection may be welded, twisted, bent, glued, tied (with suture), heat sealed to one another; or connected in any manner known in the art.

The polymer(s) useful for forming the medical device should be ones that are biocompatible and avoid irritation to body tissue. They can be either biostable or bioabsorbable. Suitable polymeric materials include without limitation polyurethane and its copolymers, silicone and its copolymers, ethylene vinyl-acetate, polyethylene terephtalate, thermoplastic elastomers, polyvinyl chloride, polyolefins, cellulosics, polyamides, polyesters, polysulfones, polytetrafluorethylenes, polycarbonates, acrylonitrile butadiene styrene copolymers, acrylics, polylactic acid, polyglycolic acid, polycaprolactone, polylactic acid-polyethylene oxide copolymers, cellulose, collagens, and chitins.

Other polymers that are useful as materials for medical devices include without limitation dacron polyester, poly(ethylene terephthalate), polycarbonate, polymethylmethacrylate, polypropylene, polyalkylene oxalates, polyvinylchloride, polyurethanes, polysiloxanes, nylons, poly(dimethyl siloxane), polycyanoacrylates, polyphosphazenes, poly(amino acids), ethylene glycol I dimethacrylate, poly(methyl methacrylate), poly(2-hydroxyethyl methacrylate), polytetrafluoroethylene poly(HEMA), polyhydroxyalkanoates, polytetrafluorethylene, polycarbonate, poly(glycolide-lactide) co-polymer, polylactic acid, poly(ε-caprolactone), poly(β-hydroxybutyrate), polydioxanone, poly(γ-ethyl glutamate), polyiminocarbonates, poly(ortho ester), polyanhydrides, alginate, dextran, chitin, cotton, polyglycolic acid, polyurethane, or derivatized versions thereof, i.e., polymers which have been modified to include, for example, attachment sites or cross-linking groups, e.g., Arg-Gly-Asp (RGD), in which the polymers retain their structural integrity while allowing for attachment of molecules, such as proteins, nucleic acids, and the like.

The polymers may be dried to increase its mechanical strength. The polymers may then be used as the base material to form a whole or part of the medical device.

Furthermore, although the invention can be practiced by using a single type of polymer to form the medical device, various combinations of polymers can be employed. The appropriate mixture of polymers can be coordinated to produce desired effects when incorporated into a medical device.

In certain preferred embodiments, the therapeutic agents described in Section 5.1.2 supra. are mixed with a polymer. Such mixture can be used to form a medical device or portions thereof. In specific embodiments, the therapeutic agent(s) constitute at least about 5%, at least about 10%, at least about 20%, at least about 30%, at least about 40%, at least about 50%, at least about 60%, at least about 70%, at least about 80%, at least about 90%, at least about 95%, at least about 97%, at least about 99% or more by weight of the polymeric compositions used to form the medical device.

In specific embodiments, the medical device comprises at least about 5%, at least about 10%, at least about 20%, at least about 30%, at least about 40%, at least about 50%, at least about 60%, at least about 70%, at least about 80%, at least about 90%, at least about 95%, at least about 97%, at least about 99% or more by weight of the polymeric material. In specific embodiments, the medical device comprises at least about 5%, at least about 10%, at least about 20%, at least about 30%, at least about 40%, at least about 50%, at least about 60%, at least about 70%, at least about 80%, at least about 90%, at least about 95%, at least about 97%, at least about 99% or more by weight of a (first) therapeutic agent, preferably paclitaxel. In specific embodiments, the medical device comprises at least about 5%, at least about 10%, at least about 20%, at least about 30%, at least about 40%, at least about 50%, at least about 60%, at least about 70%, at least about 80%, at least about 90%, at least about 95%, at least about 97%, at least about 99% or more by weight of the additional (second, third, fourth, or fifth) therapeutic agent(s).

In preferred embodiments, the medical device comprises about 0.001 μg, about 0.01 μg, about 0.1 μg, about 1 μg, 5 μg, about 10 μμg, about 15 μg, about 20 μg, about 25 μg, about 30 μg, about 35 μg, about 40 μg, about 45 μg, about 50 μg, about 60 μg, about 70 μg, about 80 μg, about 90 μg, about 100 μg, about 110 μg, about 120 μg, about 130 μg, about 140 μg, about 150 μg, about 200 μg, about 250 μg, about 300 μg, about 350 μg, about 400 μg, about 500 μg, about 600 μg, about 700 μg, about 800 μg, about 900 μg, about 1,000 μg, about 2,000 μg or more of the therapeutic agent. Preferably, the medical device comprises about 50 μg to about 200 μg paclitaxel.

In another specific embodiment, the medical device comprises about 0.001 μg, about 0.01 μg, about 0.1 μg, about 0.5 μg, about 1.0 μg, about 2.0 μg, about 3.0 μg, about 4.0 μg, about 5.0 μg, about 6.0 μg, about 7.0 μg, about 8.0 μg, about 9.0 μg, about 10.0 μg, about 15.0 μg, about 20.0 μg or more of the therapeutic agent per mm² of the surface area of the surface of the medical device. Preferably, the medical device comprises about 0.5 μg to about 5 μg paclitaxel per mm² of the surface area of the surface of the medical device.

In certain embodiments, the medical device is capable of releasing a cytostatic amount of a therapeutic agent that is effective of freezing the cell in the G1/S phase.

In one embodiment, the medical device releases about 0.1%, about 1%, about 5%, about 10%, about 15%, about 20%, about 25%, about 30%, about 35%, about 40%, about 45%, about 50%, about 55%, about 60%, about 65%, about 70%, about 75%, about 80%, about 90%, about 95% or more of the paclitaxel incorporated in the polymeric material over about 30 minutes, 1 hour, 2 hours, 6 hours, about 12 hours, about 24 hours, about 2 days, about 3 days, about 4 days, about 5 days, about 6 days, about 1 week, about 2 weeks, about 3 weeks, about 1 month, about 2 months, about 3 months, about 4 months, about 5 months, about 6 months, about 1 year, about 2 years, about 5 years, etc. Preferably, the medical device is capable of releasing about 0.1% to about 35% of the amount of the paclitaxel incorporated in the polymeric material over about 1 week to about 8 weeks. More preferably, the medical device is capable of releasing about 1% to about 15% of the amount of the paclitaxel incorporated in the polymeric material over about 4 weeks.

In another embodiment, the medical device releases about 0.001 μg, about 0.01 μg, about 0.1 μg, about 1 μg, 5 μg, about 10 μg, about 15 μg, about 20 μg, about 25 μg, about 30 μg, about 35 μg, about 40 μg, about 45 μg, about 50 μg, about 60 μg, about 70 μg, about 80 μg, about 90 μg, about 100 μg, about 110 μg, about 120 μg, about 130 μg, about 140 μg, about 150 μg, about 200 μg, about 250 μg, about 300 μg, about 350 μg, about 400 μg, about 500 μg, about 600 μg, about 700 μg, about 800 μg, about 900 μg, about 1,000 μg, about 2,000 μg or more of the therapeutic agent over about 30 minutes, 1 hour, 2 hours, 6 hours, about 12 hours, about 24 hours, about 2 days, about 3 days, about 4 days, about 5 days, about 6 days, about 1 week, about 2 weeks, about 3 weeks, about 1 month, about 2 months, about 3 months, about 4 months, about 5 months, about 6 months, about 1 year, about 2 years, about 5 years, etc. Preferably, the medical device is capable of releasing about 50 μg to about 200 μg of paclitaxel incorporated in the polymeric material over about 1 week to about 8 weeks.

In yet another embodiment, the medical device releases about 0.001 μg, about 0.01 μg, about 0.1 μg, about 0.5 μg, about 1.0 μg, about 2.0 μg, about 3.0 μg, about 4.0 μg, about 5.0 μg, about 6.0 μg, about 7.0 μg, about 8.0 μg, about 9.0 μg, about 10.0 μg, about 15.0 μg, about 20.0 μg or more of the therapeutic agent per mm² of the surface area of the surface of the medical device over about 30 minutes, 1 hour, 2 hours, 6 hours, about 12 hours, about 24 hours, about 2 days, about 3 days, about 4 days, about 5 days, about 6 days, about 1 week, about 2 weeks, about 3 weeks, about 1 month, about 2 months, about 3 months, about 4 months, about 5 months, about 6 months, about 1 year, about 2 years, about 5 years, etc. Preferably, the medical device is capable of releasing about 0.01 μg to about 0.1 μg of paclitaxel incorporated in the polymeric material over about 1 week to about 8 weeks.

In certain other embodiments, the medical device is capable of continuously releasing therapeutic agent over a period of time and thereby exposing the cells to a concentration of therapeutic agent that is effective of freezing the cell in the G1/S phase. Preferably, the concentration of therapeutic agent that the cells is exposed to is about 0.0001 ng/ml, about 0.001 ng/ml, about 0.01 ng/ml, about 0.1 ng/ml, about 1.0 ng/ml, about 10 ng/ml, about 20 ng/ml, about 30 ng/ml, about 40 ng/ml, about 50 ng/ml, about 100 ng/ml, about 200 ng/ml, about 300 ng/ml, about 400 ng/ml, about 500 ng/ml, about 600 ng/ml, about 700 ng/ml, about 800 ng/ml, about 900 ng/ml, about 1,000 ng/ml, about 2,000 ng/ml, about 3,000 ng/ml, about 4,000 ng/ml, about 5,000 ng/ml, about 10,000 ng/ml or more of the one or more therapeutic agents. Preferably, the concentration of therapeutic agent that the cells is exposed to is about 0.001 ng/ml to 10,000 ng/ml of paclitaxel. More preferably, the concentration of therapeutic agent that the cells is exposed to is about 0.01 ng/ml to 1,000 ng/ml of paclitaxel. Preferably, the concentration of therapeutic agent that the cells is exposed to is about 60 ng/ml to about 6,000 ng/ml.

Examples of the medical devices suitable for the present invention include, but are not limited to, stents, surgical staples, catheters (e.g., central venous catheters and arterial catheters), guidewires, cannulas, cardiac pacemaker leads or lead tips, cardiac defibrillator leads or lead tips, implantable vascular access ports, blood storage bags, blood tubing, vascular or other grafts, intra-aortic balloon pumps, heart valves, cardiovascular sutures, total artificial hearts and ventricular assist pumps, and extra-corporeal devices such as blood oxygenators, blood filters, hemodialysis units, hemoperfusion units and plasmapheresis units. In a preferred embodiment, the medical device is a stent.

Medical devices of the present invention include those that have a tubular or cylindrical-like portion. The tubular portion of the medical device need not to be completely cylindrical. For instance, the cross-section of the tubular portion can be any shape, such as rectangle, a triangle, etc., not just a circle. Such devices include, without limitation, stents and grafts. A bifurcated stent is also included among the medical devices which can be fabricated by the method of the present invention.

Medical devices which are particularly suitable for the present invention include any kind of stent for medical purposes which is known to the skilled artisan. Suitable stents include, for example, vascular stents such as self-expanding stents and balloon expandable stents. Preferably, the stents have openings in their sidewalls. Examples of self-expanding stents useful in the present invention are illustrated in U.S. Pat. Nos. 4,655,771 and 4,954,126 issued to Wallsten and 5,061,275 issued to Wallsten et al. Examples of appropriate balloon-expandable stents are shown in U.S. Pat. No. 5,449,373 issued to Pinchasik et al.

5.1.5 Methods of Coating the Medical Device

In the present invention, the coating composition as described in Section 5.1.1 supra. can be applied by any method to a surface of a medical device to form a coating. Examples of suitable methods are spraying, laminating, pressing, brushing, swabbing, dipping, rolling, electrostatic deposition and all modem chemical ways of immobilization of bio-molecules to surfaces. Preferably, the coating composition is applied to a surface of a medical device by spraying, rolling, laminating, and pressing. In one embodiment of the present invention, more than one coating methods can be used to make a medical device.

Furthermore, before applying the coating composition, the surface of the medical device is optionally subjected to a pre-treatment, such as roughening, oxidizing, sputtering, plasma-deposition or priming in embodiments where the surface to be coated does not comprise depressions. Sputtering is a deposition of atoms on the surface by removing the atom from the cathode by positive ion bombardment through a gas discharge. Also, exposing the surface of the device to a primer is a possible method of pre-treatment.

In certain embodiments, the medical device of the present invention is covered with one coating layer. In certain other embodiments, the medical device of the present invention is covered with more than one coating layer. In preferred embodiments, the medical device is covered with coating layers made from different coating compositions, ie., one of the coating compositions has at least one constituent or amount of a constituent that is not formed in the other coating compositions. For example, the coating can comprise a first layer and a second layer that contain different biologically active materials. Alternatively, the first layer and the second layer may contain an identical biologically active material having different concentrations. In another embodiment, the coating can comprise a first layer and a second layer that contain different therapeutic agents. In yet another embodiment, either the first layer or the second layer may be free of biologically active material.

5.2 Therapeutic Uses

The invention relates generally to the therapeutic use of pharmaceutical compositions and drug-eluting medical devices comprising the therapeutic agents as described in Section 5.1.2 supra. to prevent, treat or manage diseases or conditions associated with cell proliferation and/or migration in a subject.

As used herein, the terms “subject” and “patient” are used interchangeably. The subject can be an animal, preferably a mammal including a non-primate (e.g., a cow, pig, horse, cat, dog, rat, and mouse) and a primate (e.g., a monkey, such as a cynomolgous monkey, chimpanzee, and a human), and more preferably a human. In one embodiment, the subject can be a subject who had undergone a regimen of treatment (e.g., percutaneous transluminal coronary angioplasty (PTCA), also known as balloon angioplasty, and coronary artery bypass graft (CABG) operation).

In certain embodiments, the pharmaceutical compositions and drug-eluting medical devices may be used to inhibit the proliferation and/or migration of cells of the brain, neck, eye, mouth, throat, esophagus, chest, bone, ligament, cartilage, tendons, lung, colon, rectum, stomach, prostate, breast, ovaries, fallopian tubes, uterus, cervix, testicles or other reproductive organs, hair follicles, skin, diaphragm, thyroid, blood, muscles, bone, bone marrow, heart, lymph nodes, blood vessels, arteries, capillaries, large intestine, small intestine, kidney, liver, pancreas, brain, spinal cord, and the central nervous system. The pharmaceutical compositions and drug-eluting medical devices may be used to inhibit the proliferation and/or migration of cells of a body tissue, e.g., epithelial tissue, connective tissue, muscle tissue, and nerve tissue. Epithelial tissue covers or lines all body surfaces inside or outside the body. Examples of epithelial tissue include, but are not limited to, the skin, epithelium, dermis, and the mucosa and serosa that line the body cavity and internal organs, such as the heart, lung, liver, kidney, intestines, bladder, uterine, etc. Connective tissue is the most abundant and widely distributed of all tissues. Examples of connective tissue include, but are not limited to, vascular tissue (e.g., arteries, veins, capillaries), blood (e.g., red blood cells, platelets, white blood cells), lymph, fat, fibers, cartilage, ligaments, tendon, bone, teeth, omentum, peritoneum, mesentery, meniscus, conjunctiva, dura mater, umbilical cord, etc. Muscle tissue accounts for nearly one-third of the total body weight and consists of three distinct subtypes: striated (skeletal) muscle, smooth (visceral) muscle, and cardiac muscle. Examples of muscle tissue include, but are not limited to, myocardium (heart muscle), skeletal, intestinal wall, etc. The fourth primary type of tissue is nerve tissue. Nerve tissue is found in the brain, spinal cord, and accompanying nerve. Nerve tissue is composed of specialized cells called neurons (nerve cells) and neuroglial or glial cells.

In one embodiment, the pharmaceutical compositions and medical devices are useful for inhibiting the proliferation and/or migration of vascular smooth muscle cell, tumor cell, stromal cell, interstitial matrix surrounding vascular smooth muscle cell or immune system effector cell. In another embodiment, the pharmaceutical compositions and medical devices are capable of preventing or treating a proliferative disease, such as restenosis, stenosis, psoriasis, dermatitis, liver sclerosis, or benign prostate hyperplasia, by administering to a subject in need thereof a cytostatic amount of paclitaxel. In yet another embodiment, the pharmaceutical compositions and medical devices are capable of arresting a majority of the smooth muscle cells in the G1/S phase of the cell cycle of smooth muscle cells.

In a specific embodiment, the pharmaceutical compositions and medical devices are capable of inhibiting at least about 5%, at least about 10%, at least about 20%, at least about 30%, at least about 40%, at least about 50%, at least about 60%, at least about 70%, at least about 80%, at least about 90%, at least about 95%, at least about 97%, at least about 99% or about 100% (completely) of cell proliferation and/or migration in the cells that were exposed to the therapeutic agent, preferably paclitaxel.

In another specific embodiment, the pharmaceutical compositions and medical devices are capable of reducing at least about 5%, at least about 10%, at least about 20%, at least about 30%, at least about 40%, at least about 50%, at least about 60%, at least about 70%, at least about 80%, at least about 90%, at least about 95%, at least about 97%, at least about 99% or about 100% (completely) of the symptoms/severity/degree of restenosis, stenosis, psoriasis, dermatitis, liver sclerosis, or benign prostate hyperplasia in the subject.

In yet another specific embodiment, the pharmaceutical compositions and medical devices are capable of freezing at least about 5%, at least about 10%, at least about 20%, at least about 30%, at least about 40%, at least about 50%, at least about 60%, at least about 70%, at least about 80%, at least about 90%, at least about 95%, at least about 97%, at least about 99% or about 100% (all) smooth muscle cells at the G1/S phase of the cell cycle of smooth muscle cells.

5.2.1 Pharmaceutical Compositions

The therapeutic agents can be incorporated into a pharmaceutical composition suitable for administration. In one embodiment, the composition comprises at least two different therapeutic agents or polymer/therapeutic agent mixtures. Preferably, the composition comprises paclitaxel.

The pharmaceutical compositions may be manufactured by means of conventional mixing, dissolving, granulating, dragee-making, levigating, emulsifying, encapsulating, entrapping or lyophilizing processes. Such compositions typically comprise one or more therapeutic agents, and optionally a pharmaceutically acceptable carrier.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutically acceptable carriers include water, salt solutions, alcohol, silicone, waxes, petroleum jelly, vegetable oil, peanut oil, soybean oil, mineral oil, sesame oil, polyethylene glycols, propylene glycol, liposomes, sugars, gelatin; lactose, amylose, magnesium stearate, talc, surfactants, silicic acid, viscous paraffin, perfume oil, fatty acid monoglycerides and diglycerides, petroethral fatty acid esters, bydroxymethyl-cellulose, polyvinylpyrrolidone, and the like. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Supplementary active compounds can also be incorporated into the compositions. In one embodiment, the composition further comprises minor amounts of wetting or emulsifying agents or pH buffering agents, such as hydrochloric acid or sodium hydroxide.

The compositions can take the form of solutions, suspensions, emulsion, tablets, pills, capsules, powders, sustained-release formulations and the like, depending on its intended route of administration. Examples of routes of administration include parenteral (e.g., subcutaneous, intramuscular, intraorbital, intracapsular, intraspinal, intrastemal, intravenous, intradermal, intraperitoneal, intraportal, intra-arterial, intrathecal, transmucosal, intra-articular, and intrapleural,), transdermal (i.e., topical), epidural, and mucosal (e.g., intranasal) injection or infusion, as well as oral, inhalation, pulmonary, and rectal administration.

For parenteral administrations, the composition comprises one or more of the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampules, disposable syringes or multiple dose vials made of glass or plastic.

For topical administration, the therapeutic agents may be formulated as solutions, gels, ointments, creams, suspensions, etc. as are well-known in the art.

For injection, the therapeutic agents may be formulated in aqueous solutions, preferably in physiologically compatible buffers such as Hanks's solution, Ringer's solution, or physiological saline buffer. The solution may contain formulatory agents such as suspending, stabilizing and/or dispersing agents. In a preferred embodiment, the therapeutic agents are formulated in sterile aqueous solutions.

For intravenous administration, suitable carriers include physiological saline, bacteriostatic water, Cremophor® EL (BASF; Parsippany, N.J.) or phosphate buffered saline (PBS). In all cases, the composition must be sterile and should be fluid to the extent that easy injectability with a syringe. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

For transmucosal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories.

For transdermal administration, the therapeutic agents may be formulated into ointments, salves, gels, or creams as generally known in the art. The compounds can also be prepared in the form of suppositories (e.g., with conventional suppository bases such as cocoa butter and other glycerides) or retention enemas for rectal delivery.

In addition to the formulations described previously, the therapeutic agents may also be formulated as a depot preparation. Such long acting formulations may be administered by implantation (for example subcutaneously or intramuscularly) or by intramuscular injection. Thus, for example, the therapeutic agents may be formulated with suitable polymeric or hydrophobic materials (for example as an emulsion in an acceptable oil) or ion exchange resins, or as sparingly soluble derivatives, for example, as a sparingly soluble salt.

Additionally, the therapeutic agents may be delivered using a sustained-release system, such as semi-permeable matrices of solid polymers containing the therapeutic agent. Various forms of sustained-release materials have been established and are well known by those skilled in the art. Sustained-release capsules may, depending on their chemical nature, release the therapeutic agents for a few hours, days, weeks, months, up to over 100 days. Depending on the chemical nature and the biological stability of the therapeutic agents, additional strategies for stabilization may be employed.

As the therapeutic agents of the invention may contain charged side chains or termini, they may be included in any of the above-described formulations as the free acids or bases or as pharmaceutically acceptable salts. Pharmaceutically acceptable salts are those salts which substantially retain the biologic activity of the free bases and which are prepared by reaction with inorganic acids. Pharmaceutical salts tend to be more soluble in aqueous and other protic solvents than are the corresponding free base forms. In certain embodiments, the compositions can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The compositions of the invention will generally be used in an amount effective to achieve the intended purpose. For use to treat or prevent a proliferative disease, a therapeutically effective amount of the compositions may be administered to ameliorate or prevent the symptoms associated with the disease or condition, inhibit or reduce the growth of the hyperproliferating cells, or prolong the survival of the patient being treated. In a specific embodiment, the growth and/or number of the hyperproliferating cells is reduced by about 5%, about 10%, about 20%, about 30%, about 40%, about 50%, about 60%, about 70%, about 80%, about 90%, about 95%, about 96%, about 97%, about 98%, about 99% or about 100%.

As used herein, the term “therapeutically effective amount” refers to that amount of a therapeutic agent, preferably paclitaxel, sufficient to inhibit cell proliferation, contraction, migration, hyperactivity, or address other conditions. A therapeutically effective amount may refer to the amount of therapeutic agent sufficient to delay or minimize the onset of adverse effects and symptoms associated with cell proliferation, contraction, migration, hyperactivity. A therapeutically effective amount may also refer to the amount of the therapeutic agent that provides a therapeutic benefit in the prevention, treatment or management of certain proliferative diseases such as restenosis, stenosis, psoriasis, dermatitis, liver sclerosis, or benign prostate hyperplasia and/or the symptoms associated with the proliferative diseases. Determination of a therapeutically effective amount is well within the capabilities of those skilled in the art, especially in light of the detailed disclosure provided herein.

In certain embodiments, the therapeutically effective amount of therapeutic agent is a cytostatic amount, i.e., an amount that freezes preferably a majority of the cell in the G1/S phase or an amount that does not induce apoptotic cell death. In certain embodiments, the therapeutically effective amount of paclitaxel is about 0.001 μg, about 0.01 μg, about 0.1 μg, about 1 μg, 5 μg, about 10 μg, about 15 μg, about 20 μg, about 25 μg, about 30 μg, about 35 μg, about 40 μg, about 45 μg, about 50 μg, about 60 μg, about 70 μg, about 80 μg, about 90 μg, about 100 μg, about 110 μg, about 120 μg, about 130 μg, about 140 μg, about 150 μg, about 200 μg, about 250 μg, about 300 μg, about 350 μg, about 400 μg, about 500 μg, about 600 μg, about 700 μg, about 800 μg, about 900 μg, about 1,000 μg, about 2,000 μg or more of the therapeutic agent. Preferably, the therapeutically effective amount of paclitaxel is about 50 μg to about 200 μg paclitaxel.

In one embodiment, the therapeutically effective amount of paclitaxel is effective to arrest a majority of the smooth muscle cells of the body tissues (which is exposed to the paclitaxel) in the G1/S phase of the cell cycle of the smooth muscle cells. The amount of paclitaxel release, preferably arrests at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 90%, at least about 95%, at least about 96%, at least about 97%, at least about 98%, at least about 99% or about 100% (all) of the smooth muscle cells exposed to the paclitaxel.

In certain embodiments, the therapeutically effective amount allows the cells to be exposed to a concentration of about 0.0001 ng/ml, about 0.001 ng/ml, about 0.01 ng/ml, about 0.1 ng/ml, about 1.0 ng/ml, about 10 ng/ml, about 20 ng/ml, about 30 ng/ml, about 40 ng/ml, about 50 ng/ml, about 100 ng/ml, about 200 ng/ml, about 300 ng/ml, about 400 ng/ml, about 500 ng/ml, about 600 ng/ml, about 700 ng/ml, about 800 ng/ml, about 900 ng/ml, about 1,000 ng/ml, about 2,000 ng/ml, about 3,000 ng/ml, about 4,000 ng/ml, about 5,000 ng/ml, about 10,000 ng/ml or more of the one or more therapeutic agents. Preferably, the cells are exposed to a concentration of about 0.001 ng/ml to 1,0000 ng/ml of paclitaxel. More preferably, the cells are exposed to a concentration of about 0.01 ng/ml to 1,000 ng/ml of paclitaxel. Preferably, the cells are exposed to a concentration of about 60 ng/ml to about 6,000 ng/ml of paclitaxel.

In certain embodiments, about 5%, about 10%, about 20%, about 30%, about 40%, about 50%, about 60%, about 70%, about 80%, about 90%, about 95% or more of the therapeutic agents are released from the therapeutically effective amount over about 30 minutes, 1 hour, 2 hours, 6 hours, about 12 hours, about 24 hours, about 2 days, about 3 days, about 4 days, about 5 days, about 6 days, about 1 week, about 2 weeks, about 3 weeks, about 1 month, about 2 months, about 3 months, about 4 months, about 5 months, about 6 months, about 1 year, about 2 years, about 5 years, etc. Preferably, about 0.1% to about 35% of the therapeutically effective amount is released over about 1 week to about 8 weeks. More preferably, about 1% to 15% of the therapeutically effective amount is released over 4 weeks.

The amount of compositions to be administered may vary. The skilled artisan will appreciate that certain factors may influence the dosage required to effectively treat a subject, including but not limited to the severity of the disease or disorder, previous treatments, the general health and/or age of the subject, if other diseases are present, the manner of administration and the judgment of the prescribing physician. The treatment can be a single treatment or a series of treatments. The therapy may be repeated intermittently while symptoms detectable or even when they are not detectable. It will also be appreciated that the effective dosage of nucleic acid molecule used for treatment may increase or decrease over the course of a particular treatment. Changes in dosage may result and become apparent from the results of diagnostic and monitoring assays as described herein.

For systemic administration, a therapeutically effective dose can be estimated initially from in vitro assays. For example, a dose can be formulated in animal models to achieve a circulating concentration range that includes the IC₅₀ as determined in cell culture. Such information can be used to more accurately determine useful doses in humans.

Initial dosages can also be estimated from in vivo data, e.g., animal models, using techniques that are well known in the art. The dosage of such compositions lies preferably within a range of circulating concentrations that include the ED₅₀ with little or no toxicity. One skilled in the art could readily optimize administration to humans based on animal data.

Dosage amount and interval may be adjusted individually to provide plasma levels of the proteins which are sufficient to maintain therapeutic effect. Levels in plasma may be measured, for example, by high performance liquid chromatography.

Usual patient dosages for administration by injection range from about 0.01 to 30 mg/kg/day, preferably from about 0.1 to 10 mg/kg/day, more preferably from 0.1 to 1 mg/kg body weight. Therapeutically effective serum levels may be achieved by administering multiple doses each day. Effective doses may be extrapolated from dose-response curves derived from in vitro or animal model test systems.

The routes of administration and dosages described are intended only as a guide since a skilled practitioner will be able to determine readily the optimum route of administration and dosage for any particular patient and condition.
5.2.2 Drug-Elutin2 Medical Devices

The present invention also provides methods for treating or preventing stenosis or restenosis or other conditions involving smooth muscle cell proliferation by inserting or implanting into a subject in need thereof a medical device comprising, i.e., composed of or coated or covered with, paclitaxel. In certain embodiments, the invention relates to medical devices for insertion or implantation into a body lumen comprising smooth muscle cells, preferably vascular smooth muscle cells.

In certain embodiments, the medical device is capable of eluting a specific amount or percentage of the therapeutic agent(s) incorporated in the polymeric material of the coating. In one embodiment, the medical device elutes an amount of the therapeutic agent(s) that is capable of inhibiting a cell activity, such as protein synthesis, DNA synthesis, spindle fiber formation, cellular proliferation, cell migration, microtubule formation, microfilament formation, extracellular matrix synthesis, extracellular matrix secretion, or increase in cell volume. In one embodiment, the amount eluted is capable of altering the cellular metabolism and/or inhibiting cell proliferation and/or migration. Preferably, the cells is a vascular smooth muscle cell, tumor cell, stromal cell, interstitial matrix surrounding vascular smooth muscle cell or immune system effector cell. In one embodiment, the amount eluted allows for cellular repair and matrix production. Preferably, the amount eluted is cytostatic and does not kill the cell (by either the apoptotic or necrotic pathway). More preferably, the amount eluted is capable of arresting a majority of the smooth muscle cells in the G1/S phase of the cell cycle, without killing the cell.
5.2.3 Combination Therapy

The present invention is useful alone or in combination with other treatment modalities. The pharmaceutical compositions and medical devices of the invention can be administered to a subject, sequentially or simultaneously, with surgery, standard and experimental chemotherapies, hormonal therapies, biological therapies, immunotherapies, radiation therapies, embolization, and/or chemoembolization therapies for the treatment or prevention of diseases or conditions associated with hyperproliferating cells.

The pharmaceutical compositions and medical devices of the invention can also be administered to a subject, sequentially or simultaneously, with a further therapeutic agent which may be the same as or different to the therapeutic agent. In one embodiment, the further therapeutic agent may be one or more immunotherapeutic agents, such as antibodies and immunomodulators, which include, but are not limited to, HERCEPTIN®, RITUXAN(®, OVAREX™, PANOREX®, BEC2, IMC-C225, VITAXIN™, CAMPATH® I/H, Smart MI95, LYMPHOCIDE™, Smart I D10, ONCOLYM™, rituximab, gemtuzumab, or trastuzumab.

### 6. EXAMPLE

6.1 Materials And Methods
6.1.1 Cell Lines and Reagents

Human aortic smooth muscle cells (hSMC) were purchased from Cambrex, Inc (CC-2571, San Diego, Calif.). The cells were grown in SmGM-2(D - Smooth Muscle Growth Medium-3 (Cambrex, San Diego, Calif.). Jurkat cells, a human leukemia cell line, were obtained from American Type Culture Collection (Manassas, Va.) and served as a control. Paclitaxel was investigated in two forms, paclitaxel (PTX) (Hauser, Boulder, CO) and Taxol® in Cremophor® EL (Bristol-Myers-Squibb, Princeton, N.J.). The former was diluted in ethanol and the working solution was prepared in fresh SmGM-2 medium. The use of Taxol® was limited to the cell counting assay; both paclitaxel and Taxol were equipotent. doxorubicin (DOX) (Adriamycin®) and phorbol myristate acetate (PMA) were obtained from Sigma (St. Louis, Mo.) and dissolved in DMSO as a 2 mg/ml stock solution.
6.1.2 MTT Assay for Cell Proliferation

2,000 cells per well were plated in 96-well flat bottom plates and then exposed to paclitaxel (PTX) at concentrations from 0.04 to 100 ng/ml. After either 3 or 4 days, 20 μL of 5 mg/ml MTT (3,[4,5-dimethylthiazol-2-yl]-2,5-diphenyltetrazolium bromide Sigma, St. Louis) was added to each well for 4 hrs. After removal of the medium, 170 μL of dimethyl sulfoxide (DMSO) was added to each well to dissolve the formazan crystals formed from the MTT. The absorbance at 540 nm was determined using a Biokinetics plate reader (Bio-Tek Instruments, Inc, Winooski, Vt.) as previously described (Giannakakou P. et al. Low concentrations of paclitaxel induce cell type-dependent p53, p21 and G1/G2 arrest instead of mitotic arrest: molecular determinants of paclitaxel-induced cytotoxicity. Oncogene. 2001 Jun. 28;20(29):3806-13). Triplicate wells were assayed for each condition and standard deviations were determined. Cells without PTX treatment served as controls.

6.1.3 Cell Counting and Viability Assay

Human smooth muscle cells (10⁴ to 2×10⁴ cells) in 1 ml of SmGM-2® medium were plated in 24-well plates, and were treated with PTX for up to 8 days. PTX concentrations ranged from 0.1 to 5000 ng/ml. After the indicated incubation time, cells were harvested by trypsinization 0.25% trypsin (Gibco BRL) for 5 min and counted in triplicate on a Coulter Z1 cell counter (Hialeah, Fla., USA). Cells without PTX treatment served as controls. In addition, cells were incubated with trypan blue and the numbers of blue (dead) cells and transparent (live) cells were counted in a hemocytometer. Data were reported as the percentage of live cells with respect to the control, which was either with vehicle only (at the designated time point) or at time=0. In the subsequent study to investigate the effects of paclitaxel on hSMC proliferation during an 8 day period, 2 and 12 ng/ml of PTX concentrations were used, which bracketed the near-maximal inhibition of hSMC proliferation.
6.1.4 Flow Cytometry for Cell Cycle Analysis

Human smooth muscle cells were exposed to PTX at concentrations of 2, 6, 12, and 60 ng/ml for 2 days. The cells were then harvested by trypsinization, washed with PBS and resuspended in 75% ethanol in PBS and kept at 4° C. for at least 30 minutes. Prior to analysis, cells were washed again with PBS and resuspended and incubated for 30 min in propidium iodide staining solution containing 1 mg/ml RNAse A and 0.05 mg/ml propidium iodide (Sigma, St. Louis), as previously described (Giannakakou P. et al. Low concentrations of paclitaxel induce cell type-dependent p53, p21 and G1/G2 arrest instead of mitotic arrest: molecular determinants of paclitaxel-induced cytotoxicity. Oncogene. 2001 Jun. 28;20(29):3806-13). The suspension was then analyzed on a Becton Dickinson FACScan.

DNA content was measured using a FACScan flow cytometer (Becton Dickinson Immunocytometry Systems, San Jose, Calif.). To calculate percentage of cells in respective phases of the cell cycle the DNA content frequency histograms were deconvoluted using the MultiCycle software (Phoenix Flow Systems, San Diego, Calif.).
6.1.5 Immunoblot Analysis Cell Cycle and Apoptosis Markers

Cells were lysed and soluble proteins were harvested in TNES buffer (50 mM TrisHCl pH 7.5, 100 mM NaCl, 2 mM EDTA, 1 mM sodium orthovanadate, and 1% (v/v) NP40, Sigma, St. Louis, Mo.) containing protease inhibitors, as previously described (Blagosklonny M. V. et al. Mitogen activated protein kinase pathway is dispensible for microtubule-active drug-induced Raf-1/Bcl-2 phosphorylation and apoptosis in leukemia cells. Leukemia 1999;13:1028-1036). Proteins were resolved with SDS-PAGE or NuPAGE 4-12% Bis-Tris gel with MOPS running buffer (NOVEX, San Diego, Calif.) according to manufacturer's instructions. Immunoblotting was performed using rabbit polyclonal anti-PARP and monoclonal mouse anti-caspase-9 (Upstate Biotechnology, Lake Placid, N.Y.), monoclonal mouse anti-p21 and anti-p53 (Oncogene Res., Calbiochem), monoclonal mouse antihuman tubulin and actin (Sigma, St. Louis, Mo.), monoclonal mouse anti-human caspase-8 (Pharmingen, San Diego, Calif.) and caspase-3 (Transduction Laboratories, Lexington, Ky.), polyclonal antibodies against 89 kDa cleaved PARP fragment (Promega, Madison, Wis.), and anti-GAPDH antibodies (Sigma, St. Louis), used to confirm an equal loading. Doxorubicin (DOX), a strong inducer of p53, was used as a positive control. Phorbol 12-myristate 13-acetate (PMA) (Sigma, St. Louis, Mo.) was used as a negative control for p53 induction.
6.1.6 Mitotic Index

Cells were incubated with 2, 6, 12 and 60 ng/ml PTX for 2 days. Cells were subsequently trypsinized, washed with PBS, pelleted onto glass slides in a cytocentrifuge, fixed with 90% ethanol/ 10% glacial acetic acid and stained with DAPI (4,6-daminidino-2-phenylindole, Molecular Probes, Eugene, Oreg.) as described previously (An W. G. et al. Protease inhibitor-induced apoptosis: accumulation of wt p53, p21WAF1/CIP1, and induction of apoptosis are independent markers of proteasome inhibition. Leukemia. 2000 Jul.;14(7):1276-83). Nuclei were visualized by UV microscopy to identify the cells in interphase and mitotic stages.
6.1.7 Cellular and Nuclear Morphology

To analyze hSMC morphology, cells were incubated with 2, 6, and 60 ng/ml of PTX for up to 21 days. The cells were cytocentrifuged, fixed in 1% formaldehyde in phosphate-buffered saline (PBS) for 15 min, then in 70% ethanol, stained in a solution containing 1 g/ml of DAPI (Molecular Probes, Inc., Eugene, Oreg.) and 20 μg/ml of sulforhodamine 101 (Molecular Probes) in PBS and inspected under UV microscope (Nikon Microphot microscope) for MN cells to determine the effect of PTX on the typical spindle-shaped morphology of hSMC and nuclear morphology. Alternatively, DNA was stained with Rhodamine (red) and cytoplasmic proteins with FITC (green). These cells were visualized as red (nuclei) and green (cytoplasm) using the laser-scanning cytometer (LSC), a microscope-based cytofluorometer. Images were captured of the laser-excited fluorescence emitted from fluorochromed cells on a microscope slide.
6.2 Results
6.2.1 Inhibition of Proliferation of SMC by PTX

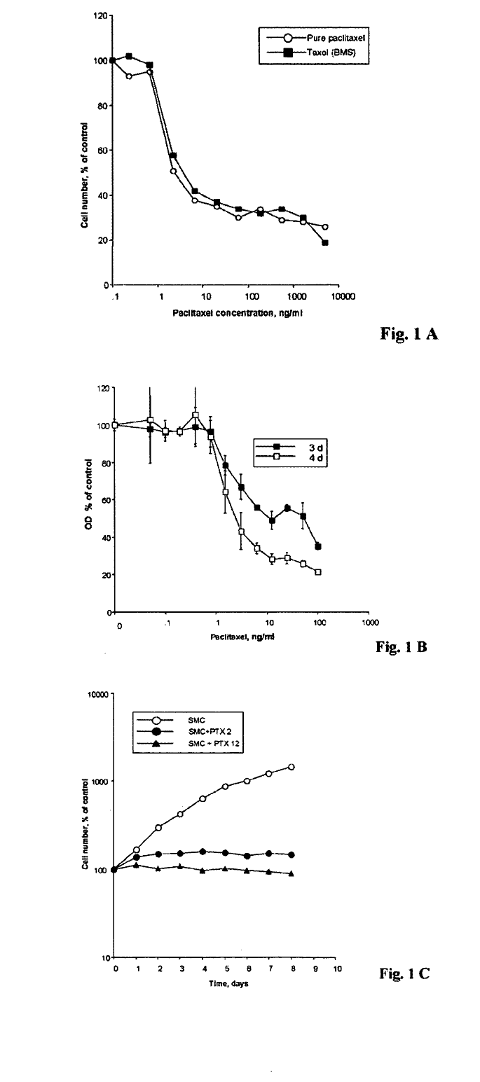

Using the MTT and Cell Counting assays, PTX at doses as low as 1 ng/ml, inhibited proliferation of hSMC by 60% of the vehicle control up to 5 days (FIGS. 1A and 1B). Identical results were obtained with 2 different formulations of PTX (PTX in ethanol and Taxol® in Cremophor® EL) (FIG. 1A). Growth inhibition of hSMC by PTX was dose-dependent at concentrations up to 12 ng/ml (low concentrations), above which no additional inhibition occurred up to 5000 ng/ml.

In the subsequent 8-day studies with 2 and 12 ng/ml of PTX, cell proliferation was completely blocked at 12 ng/ml in comparison with controls (FIG. 1C). No cell death was observed by trypan-blue exclusion staining, and the number of cells remained constant indicating that PTX arrested proliferation of hSMC but did not kill the cells.

6.2.2 Induction of Primary G1 Arrest in SMC by Paclitaxel

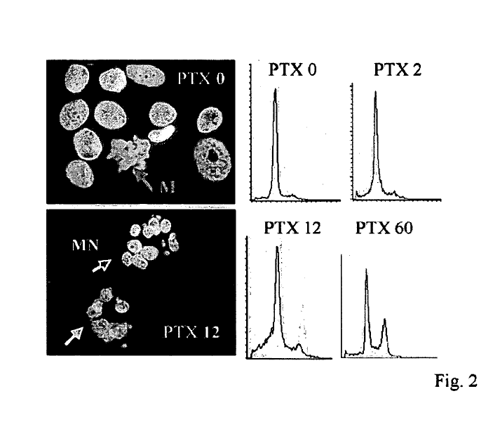

DAPI staining of DNA in control untreated hSMC revealed normal interphase nuclei (97-99%), with a few mitotic figures (1-3% of cells, FIG. 2A, upper panel), and no MN cells. In the presence of 12 ng/ml PTX for 2 days, mitotic cells formed, became rounded and detached from the plastic. However, these cells then re-attached indicating mitotic exit: 30-40% of these cells were MN (FIG. 2A, lower panel).

Primary G1 arrest in SMC at low concentrations of PTX was demonstrated by flow cytometry at 2 and 12 ng/ml (FIG. 2), where maximal proliferation inhibition was achieved (FIG. 1). At higher PTX concentrations (60 ng/ml), there was an increase in the 4C peak as measured by flow cytometry. This peak indicates either a G2/M arrest or the formation of MN cells post mitotic exit. Given that there was a large population of cells arrested in G1 and the cell cycle distribution was seemingly unperturbed, the flow cytometry peak indicates the cells were MN and not arrested in G2 (FIG. 2B).
6.2.3 PTX-induced 2C and 4C MN Cells

Following treatment with low (6 ng/ml) or high concentrations (60 ng/ml) of PTX, similar percentages of MN cells and widely differing percentages of 4C cells were observed (FIG. 3). At 60 ng/ml PTX, the percentages of 4C DNA cells (by flow cytometry) and MN cells (by nuclei staining) coincided, indicating that SMC exited mitosis to form MN 4C DNA cells, without cytokinesis. After treatment with 6 ng/ml PTX, MN cells had 2C DNA content (FIG. 3), indicating cell division. Given that MN cells can be formed only from 4C cells during abnormal mitosis, this indicates that cells managed to divide, leading to 2C DNA content and multinuclear morphology, as shown in FIG. 3B. Two groups of micronuclei (blue arrows) pulled DNA (red arrow) to form 2C DNA (normal DNA content) MN cells. The amount of DNA in the daughter cells could be slightly more than 2C or less than 2C in each of the daughter cell (2N+). The widening that was observed of the flow cytometry 2C DNA peaks following treatment with PTX (FIGS. 2 and 3) is due to this uneven distribution of chromosomes during cell division.
6.2.4 Retention of Normal SMC Morphology After Exposure to PTX

SMC have a typical spindle-shaped morphology with elongated processes as seen under light microscopy (FIG. 3A, control). PTX treatment did not change the appearance of the SMC cells, although fewer cells were detected following PTX treatment for 3 days (FIG. 3A, 60 ng/ml PTX). Under high magnification, analysis of the nuclei of these cells showed that 60-70% of the cells were arrested in the interphase and 30-40% of the cells were MN, indicative of mitotic exit after mitotic arrest. Micronuclei were distributed within the cytoplasmic body of the cells (FIG. 3B). The lowest concentration of PTX tested (2 ng/ml) caused the appearance of lobed nuclei, which are a mark of mitotic dysfunction.
6.2.5 Induction of p53 and P21 by PTX

After 1 day of treatment with increasing concentrations of PTX, a dose dependent increase in levels of p53 and p21 was seen in PTX treated hSMC (FIG. 5). Detection levels with the highest doses, 20 and 60 ng/ml PTX, were similar to those seen with 400 ng/ml doxorubicin, the positive control for p53 and p21 induction. Given that p21 inhibits cyclin dependent kinases (cdks), necessary for G1/S transition, this finding suggests the p21-mediated inhibition of cdks as a possible mechanism for G1 arrest of hSMC by PTX.
6.2.6 Lack of Apoptosis in SMC Post-exposure to PTX

Prolonged cell arrest during mitosis can lead to apoptosis. The presence of MN cells seen in our studies, indicative of a successful mitotic exit, is a marker of apoptosis avoidance.³⁷ Lack of apoptosis in hSMC was further confirmed by the lack of cleavage of PARP and caspase-3, -9, -8, even after 2 days of PTX treatment (FIG. 6). The cleavage of PARP and activation of caspases 3, 8, and 9 are indicative of apoptotic cells. Control Jurkat cells treated with 12 ng/ml of PTX for 16 hours showed disappearance (activation) of the caspase-3, -8 and -9 bands and cleavage of PARP, with appearance of an 85-kDa fragment (FIG. 6). In comparison, SMC expressed low levels of caspases. This finding further confirmed that prolonged exposure to PTX concentrations as high as 300 ng/ml did not result in hSMC apoptosis.

6.2.7 Long Term Survival of SMC

Following 21 days of treatment with 60 ng/ml PTX, SMC cells were viable and adherent to the plastic (FIG. 7). There was no change in numbers of either MN cells or 2C compared with that seen for the short-term treatment (FIGS. 2 and 3). Analysis of cell culture indicated that most cells were with 2C DNA content (FIG. 7).
6.3 Discussion

Apoptotic cell death is considered to be the principle mechanism of cellular inhibition for several anti-neoplastic drugs, including PTX. However, in certain neoplastic cell lines, PTX does not cause apoptosis but rather inhibits proliferation by several other mechanisms depending on the cell type and concentration of paclitaxel (Giannakakou et al. supra. 2001; Panvichian R. et al. Paclitaxel-associated multimininucleation is permitted by the inhibition of caspase activation: a potential early step in drug resistance. Cancer Res. 1998 Oct. 15;58(20):4667-72; Merlin J. L. et al. Resistance to paclitaxel induces time-delayed multinucleation and DNA fragmentation into large fragments in MCF-7 human breast adenocarcinoma cells. Anticancer Drugs. 2000 Apr.;1 1(4):295-302; Broker L. E. et al. Late activation of apoptotic pathways plays a negligible role in mediating the cytotoxic effects of discodermolide and epothilone B in non-small cell lung cancer cells. Cancer Res. 2002 Jul. 15;62(14):4081-8; Blagosklonny M. V. et al. supra. 2002; Roberts J. R. et al. Development of polyploidization in taxol-resistant human leukemia cells in vitro. Cancer Res. 1990 Feb. 1;50(3):710-6; Weitzel D. H. et al. Differential spindle assembly checkpoint response in human lung adenocarcinoma cells. Cell Tissue Res. 2000 Apr.;300(1):57-65; Lanzi C. et al. Cell cycle checkpoint efficiency and cellular response to paclitaxel in prostate cancer cells. Prostate. 2001 Sep. 15;48(4):254-64). This study demonstrated that PTX at concentrations as high as 1,000 ng/ml inhibited the proliferation of SMC without causing cell death. This lack of cytotoxicity is similar to previously published results that found that hSMC were viable following 8 days of incubation with 1 μg/ml PTX, even though this high dose of PTX caused tubulin polymerization. (Axel D. I. et al. PTX inhibits arterial smooth muscle cell proliferation and migration in vitro and in vivo using local drug delivery. Circulation 1997;96:636-645).

In the current study, the detailed mechanism of growth inhibition of hSMC caused by doses of PTX relevant to local, stent-based delivery was investigated. After exposure to PTX, hSMC underwent a transient mitotic arrest, exited mitosis and formed viable MN cells, and remained viable after 21 days of treatment. The cell cultures included two types of cells: MN cells, which are consistent with a secondary postmitotic arrest, and a larger population of cells with 2C DNA content and one nucleus consistent with a primary G1 arrest (FIGS. 2 and 3). PTX also induced p53 and p21 in hSMC. It is known that p21 induction is essential for G1 arrest caused by microtubule dysfunction (Cross S. M. et al. A p53-dependent mouse spindle checkpoint. Science. 1995 Mar 3;267(5202):1353-6; Lanni J. S. et al. Characterization of the p53-dependent postmitotic checkpoint following spindle disruption. Mol Cell Biol. 1998 Feb.;18(2):1055-64; Mantel C. R. et al. P21waf-1-Chk1 pathway monitors G1 phase microtubule integrity and is crucial for restriction point transition. Cell Cycle. 2002 Sep.-Oct.;1(5):327-36). Additionally, p21 protects cells by inhibiting their mitotic entry (Li W. et al. Overexpression of p21(waf1) decreases G2-M arrest and apoptosis induced by paclitaxel in human sarcoma cells lacking both p53 and functional Rb protein. Mol Pharmacol. 1999 Jun.;55(6): 1088-93), as well as accelerating mitotic exit (Barboule N. et al. Involvement of p21 in mitotic exit after paclitaxel treatment in MCF-7 breast adenocarcinoma cell line. Oncogene. 1997 Dec. 4;15(23):2867-75). Mitotic exit and primary and secondary G1 arrest, as seen in this study may explain the hSMC resistance to PTX-induced apoptosis.

In apoptosis-prone cells such as Jurkat cells, PTX caused activation of caspase-9, caspase-3, and caspase-8 and PARP cleavage, markers of apoptosis (FIG. 6). In contrast, PTX did not induce apoptosis in hSMC. This finding may be explained with two mechanisms. First, hSMC may not undergo apoptosis because they express constitutively low levels of pro-caspases. Second, since prolonged mitotic arrest can lead to apoptosis, the mitotic exit and secondary G1 arrest shown in this study prevents the cells from undergoing apoptosis. A similar phenomenon has been demonstrated in HeLa cells treated simultaneously with PTX and z-VAD-fmk, a caspase inhibitor, establishing a connection between PTX resistance, lack of caspase activation, and the MN phenotype (Panvichian R. et al. PTX-associated multimininucleation is permitted by the inhibition of caspase activation: a potential early step in drug resistance. Cancer Res 1998;58:4667-4672; Roberts J. R. et al. Development of polyploidization in taxol resistant human leukemia cells in vitro. Cancer Res 1990;50:710-716; Weitzel D. H. et al. Differential spindle assembly checkpoint response in human lung adenocarcinoma cells. Cell Tissue Res 2000;300:57-65; Lanzi C. et al. Cell cycle checkpoint efficiency and cellular response to PTX in prostate cancer cells. Prostate 2001;48:254-264). Therefore, it may be not surprising that hSMC, which have low caspase levels and can overcome mitotic arrest, do not undergo apoptosis following PTX treatment.

When cells exit mitosis without chromosomal segregation, they form nuclear membranes around each group of chromosomes, manifested as micronuclei. It has been established in this study that MN cells obtained after treatment with PTX may contain a tetraploid amount of DNA (4C). When studying the distribution of 2C, 4C and MN cells at varying PTX concentrations, we observed an interesting phenomenon. Although MN cells were produced by both low (6 ng/ml) and high concentrations (60 ng/ml) of PTX, only high concentrations produced 4C cells. At low concentrations of PTX, most MN cells had 2C DNA content. This indicates that cells managed to divide, although without proper chromosome segregation (FIG. 8). This leads to 2C DNA content and multinuclear morphology. Given that cells have two copies of genes and chromosomes, loss or acquisition of one copy does not affect cell survival.

Increased DNA content occurs naturally in arterial SMC (Hixon M. L. Vascular Smooth Muscle Polyploidization: From mitotic checkpoints to hypertension. Cell Cycle 2003;2: 105-110), causing neither cell death nor tumors. Polyploidy is normally seen where cellular hypertrophy occurs with various cell types such as terminally differentiated cardiac myocytes, non-terminally differentiated hepatocytes, and smooth muscle cells Devlin A. M. et al. The effect of perindopril on vascular smooth muscle polyploidy in stroke-prone spontaneously hypertensive rats. J Hypertens 1995; 13:211-218; Engelmann G. et al. Age-related changes in ploidy levels and biochemical parameters in cardiac myocytes isolated from spontaneously hypertensive rats. Circ Res 1986;58:137-147; Brodsky W. et al. Cell polyploidy: its relation to tissue growth and function. Int Rev Cytol 1977;50:275-332; Owens G. K. Control of hypertrophic versus hyperplastic growth of vascular smooth muscle cells. Am J Physiol. 1989;257:H1755-H1765). Polyploidy in human artery wall smooth muscle cells ranged from 1 to 7% of the hSMC population depending on the age of the subject (Barrett T. B. et al. Polyploid nuclei in human artery wall smooth muscle cells. Proc Natl Acad Sci USA 1983;80:882-885). While polyploidy can lead to altered gene expression in yeast (Galitski T. et al. Ploidy regulation of gene expression. Science 1999;285:251-254) the functional significance of polyploidy is unknown in hSMC. Further research in this area will contribute to a better understanding of the significance of polyploidy in hSMC.
6.4 Conclusion

These in vitro studies demonstrated that paclitaxel effectively inhibited the proliferation of hSMC without inducing apoptosis. Additionally, it was shown for the first time that paclitaxel at all concentrations examined resulted in primary and post-mitotic G 1 arrest in hSMC. The cells maintained their viability even after exposure to paclitaxel for up to 21 days. All of these findings indicate that paclitaxel, at concentrations relevant to stent-based delivery of anti-restenotic agents, is cytostatic rather than cytotoxic.

### 7. EXAMPLE

7.1 Methods

Stents of different lengths (16 mm and 32 mm) were coated with 1 μg of paclitaxel per mm² stent surface area. Type 1 stents had a coating consisting of 8.8% of paclitaxel by weight and 91.2% styrene-isobutylene copolymer by weight. Type 2 stents had a coating consisting of 25% of paclitaxel by weight and 75% styrene-isobutylene copolymer by weight.

The stents were incubated in a phosphate buffered release medium and the cumulative amount of paclitaxel eluted from the stents were measured on days 2, 4, 7 and 10 using HPLC methods.

7.2 Results

The amounts of paclitaxel released into the medium as measured on days 2, 4, 7 and 10 are shown in Tables 1 and 3.

The percentages of paclitaxel released as measured on days 2, 4, 7 and 10 are shown in Tables 2 and 4 and were calculated using the following formula:

PTX cumulative % released =PTX cumulative released/amount loaded.

TABLE 1
Total amount of PTX released from Type 1 stents

| | PTX Cumulative Released (μg) | | | |
|---|---|---|---|---|
| Stent length | Day 2 | Day 4 | Day 7 | Day 10 |
| 16 mm | 0.48 ± 0.06 | 0.73 ± 0.06 | 0.97 ± 0.11 | 1.29 ± 0.11 |
| 32 mm | 1.00 ± 0.18 | 1.55 ± 0.24 | 2.04 ± 0.20 | 2.64 ± 0.23 |

TABLE 2
Total percentage of PTX released from Type 1 stents

| | PTX Cumulative Released (%) | | | |
|---|---|---|---|---|
| Stent length | Day 2 | Day 4 | Day 7 | Day 10 |
| 16 mm | 0.44 ± 0.05 | 0.70 ± 0.08 | 0.93 ± 0.15 | 1.20 ± 0.10 |
| 32 mm | 0.48 ± 0.09 | 0.75 ± 0.11 | 0.98 ± 0.10 | 1.27 ± 0.11 |

TABLE 3
Total amount of PTX released from Type 2 stents

PTX Cumulative Released (μg)

| Stent length | Day 2 | Day 10 |
|---|---|---|
| 16 mm | 9.75 ± 1.64 | 12.98 ± 1.13 |
| 32 mm | 21.76 ± 3.33 | 27.35 ± 4.99 |

TABLE 4
Total percentage of PTX released from Type 2 stents

PTX Cumulative Released (%)

| Stent length | Day 2 | Day 10 |
|---|---|---|
| 16 mm | 9.03 ± 1.51 | 12.02 ± 1.05 |
| 32 mm | 10.42 ± 1.57 | 13.12 ± 2.35 |

8. Equivalents

The present invention is not to be limited in scope by the specific embodiments described which are intended as single illustrations of individual aspects of the invention, and functionally equivalent methods and components are within the scope of the invention. Indeed, various modifications of the invention, in addition to those shown and described herein, will become apparent to those skilled in the art from the foregoing description and accompanying drawings using no more than routine experimentation. Such modifications and equivalents are intended to fall within the scope of the appended claims.

All publications, patents and patent applications mentioned in this specification are herein incorporated by reference into the specification to the same extent as if each individual publication, patent or patent application was specifically and individually indicated to be incorporated herein by reference.

Citation or discussion of a reference herein shall not be construed as an admission that such is prior art to the present invention.

1. A medical device for implantation into a body lumen comprising smooth muscle cells, the medical device comprising a surface and a coating disposed on the surface;
wherein the coating comprises a polymeric material incorporating paclitaxel; and
wherein the coating is capable of releasing an amount of the paclitaxel incorporated in the polymeric material that is effective to arrest the smooth muscle cells of the body lumen exposed to the paclitaxel in the G1/S phase of the cell cycle of the smooth muscle cells.

2. The medical device of claim 1, wherein about 90% to about 100% of the smooth muscle cells are arrested in the G1/S phase.

3. The medical device of claim 1, wherein the amount of paclitaxel incorporated in the polymeric material is about 0.5 μg to about 5 μg per mm² of the surface area of the surface.

4. The medical device of claim 3, wherein the amount of paclitaxel incorporated in the polymeric material is about 1 μg per cm² of the surface area of the surface.

5. The medical device of claim 1, wherein the amount of paclitaxel released exposes the cells to a concentration of paclitaxel that is about 0.001 ng/ml to about 10,000 ng/ml.

6. The medical device of claim 5, wherein the amount of paclitaxel released exposes the cells to a concentration of paclitaxel that is about 0.01 ng/ml to about 1,000 ng/ml.

7. The medical device of claim 5, wherein the amount of paclitaxel released exposes the cells to a concentration of paclitaxel that is about 60 ng/ml to about 6,000 ng/ml.

8. The medical device of claim 1, wherein the polymeric material is biostable.

9. The medical device of claim 1, wherein the polymeric material comprises a styrene-isobutylene copolymer.

10. The medical device of claim 1, wherein the medical device is a stent.

11. The medical device of claim 1, wherein the polymeric material further comprises a biologically active material.

12. A method for treating stenosis or restenosis comprising inserting or implanting the medical device of claim 1 into a subject.

13. A medical device for implantation into a body lumen comprising smooth muscle cells, the medical device comprising a surface and a coating disposed on the surface;
wherein the coating comprises a polymeric material incorporating paclitaxel;
wherein the coating is capable of releasing about 0.001 μg to about 20 μg of the paclitaxel incorporated in the polymeric material per mm² of the surface area of the surface over about 1 week to about 8 weeks.

14. The medical device of claim 13, wherein the coating is capable of releasing about 0.01 μg to about 0.1 μg of the paclitaxel incorporated in the polymeric material per mm² of the surface area of the surface over about 4 weeks.

15. The medical device of claim 13, wherein the amount of paclitaxel released from the coating is effective to arrest the smooth muscle cells exposed to the paclitaxel in the G1/S phase of the cell cycle.

16. The medical device of claim 15, wherein about 90% to about 100% of the smooth muscle cells are arrested in the G1/S phase.

17. The medical device of claim 13, wherein the amount of paclitaxel incorporated in the polymeric material is about 0.5 μg to about 5 μg per mm² of the surface area of the surface.

18. The medical device of claim 17, wherein the amount of paclitaxel incorporated in the polymeric material is about 1 μg per cm² of the surface area of the surface.

19. The medical device of claim 13, wherein the amount of paclitaxel released exposes the cells to a concentration of paclitaxel that is about 0.001 ng/ml to about 10,000 ng/ml.

20. The medical device of claim 19, wherein the amount of paclitaxel released exposes the cells to a concentration of paclitaxel that is about 0.01 ng/ml to about 1,000 ng/ml.

21. The medical device of claim 19, wherein the amount of paclitaxel released exposes the cells to a concentration of paclitaxel that is about 60 ng/ml to about 6,000 ng/ml.

22. The medical device of claim 13, wherein the polymeric material is biostable.

23. The medical device of claim 13, wherein the polymeric material comprises a styrene-isobutylene copolymer.

24. The medical device of claim 13, wherein the medical device is a stent.

25. The medical device of claim 13, wherein the polymeric material further comprises a biologically active material.

26. A method for treating stenosis or restenosis comprising inserting or implanting the medical device of claim 13 into a subject.

27. A medical device for implantation into a body lumen comprising smooth muscle cells, the medical device comprising a surface and a coating disposed on the surface;
wherein the coating comprises a polymeric material incorporating an amount of paclitaxel;
wherein the coating is capable of releasing about 0.1% to about 35% of the amount of the paclitaxel incorporated in the polymeric material over about 1 week to about 8 weeks.

28. The medical device of claim 27, wherein the coating is capable of releasing about 1% to about 15% of the amount of the paclitaxel incorporated in the polymeric material over about 4 weeks.

29. The medical device of claim 27, wherein the amount of paclitaxel released from the coating is effective to arrest the smooth muscle cells in the G1/S phase of the cell cycle.

30. The medical device of claim 29, wherein about 90% to about 100% of the smooth muscle cells are arrested in the G1/S phase of the cell cycle.

31. The medical device of claim 27, wherein the amount of paclitaxel incorporated in the polymeric material is about 0.5 μg to about 5 μg per mm of the surface area of the surface.

32. The medical device of claim 31, wherein the amount of paclitaxel incorporated in the polymeric material is about 1 μg per cm² of the surface area of the surface.

33. The medical device of claim 27, wherein the amount of paclitaxel released exposes the cells to a concentration of paclitaxel that is about 0.001 ng/ml to about 10,000 ng/ml.

34. The medical device of claim 33, wherein the amount of paclitaxel released exposes the cells to a concentration of paclitaxel that is about 0.01 ng/ml to about 1,000 ng/ml.

35. The medical device of claim 33, wherein the amount of paclitaxel released exposes the cells to a concentration of paclitaxel that is about 60 ng/ml to about 6,000 ng/ml.

36. The medical device of claim 27, wherein the polymeric material is biostable.

37. The medical device of claim 27, wherein the polymeric material comprises a styrene-isobutylene copolymer.

38. The medical device of claim 27, wherein the medical device is a stent.

39. The medical device of claim 27, wherein the polymeric material further comprises a biologically active material.

40. A method for treating stenosis or restenosis comprising inserting or implanting the medical device of claim 27 into a subject.

41. A method for treating a proliferative disease in a subject, comprising administering a therapeutically effective amount of paclitaxel into a body lumen of the subject that comprises smooth muscle cells, wherein the therapeutically effective amount of paclitaxel is effective to arrest the smooth muscle cells of the body lumen in the G1/S phase of the cell cycle.

42. The method of claim 41, wherein about 90% to about 100% of the smooth muscle cells are arrested in the G1/S phase of the cell cycle.

43. The method of claim 41, wherein the therapeutically effective amount of paclitaxel exposes the cells to a concentration of paclitaxel that is about 0.001 ng/ml to about 10,000 ng/ml.

44. The method of claim 43, wherein the therapeutically effective amount of paclitaxel exposes the cells to a concentration of paclitaxel that is about 0.01 ng/ml to about 1,000 ng/ml.

45. The method of claim 43, wherein the therapeutically effective amount of paclitaxel exposes the cells to a concentration of paclitaxel that is about 60 ng/ml to about 6,000 ng/ml.

46. The method of claim 41, wherein the proliferative disease is restenosis, stenosis, psoriasis, dermatitis, liver sclerosis, or benign prostate hyperplasia.

47. The method of claim 41, wherein the paclitaxel is administered to the subject by parenteral, subcutaneous, intramuscular, intraorbital, intracapsular, intraspinal, intrastemal, intravenous, intradermal, intraperitoneal, intraportal, intra-arterial, intrathecal, transmucosal, intra-articular, and intrapleural, transdermal, topical, epidural, mucosal, intranasal injection or infusion, or oral, inhalation, pulmonary or rectal administration.

48. A method of arresting smooch muscle cells in the G1/S phase comprising exposing the cells to a concentration of paclitaxel that is about 0.001 ng/ml to about 10,000 ng/ml.

49. The method of claim 48, wherein the cells are exposed to a concentration of paclitaxel that is about 0.01 ng/ml to about 1,000 ng/ml.

50. The method of claim 48, wherein the cells are exposed to a concentration of paclitaxel that is about 60 ng/ml to about 6,000 ng/ml.

51. The method of claim 48, wherein about 90% to about 100% of the smooth muscle cells are arrested in the G1/S phase.
